(19) United States
(12) Patent Application Publication
(10) Pub. No.: US 2024/0139196 A1
(43) Pub. Date: May 2, 2024

(54) COMBINATION TREATMENTS IN DIFFICULT TO TREAT PATIENTS WITH ADVANCED AND/OR METASTATIC TROP-2 OVEREXPRESSING CANCERS

(71) Applicant: G1 Therapeutics, Inc., Research Triangle Park, NC (US)

(72) Inventors: Andrew Beelen, Cary, NC (US); Rajesh Kumar Malik, Chapel Hill, NC (US); John Seung-Hoon Yi, Durham, NC (US); Curt Douglas Wolfgang, Durham, NC (US)

(73) Assignee: G1 Therapeutics, Inc., Research Triangle Park, NC (US)

(21) Appl. No.: 18/394,512

(22) Filed: Dec. 22, 2023

Related U.S. Application Data
(63) Continuation of application No. PCT/US2022/035995, filed on Jul. 1, 2022.
(60) Provisional application No. 63/217,716, filed on Jul. 1, 2021.

(51) Int. Cl. A61K 31/519; A61K 31/4375; A61K 47/68; A61P 35/00; C07K 16/30

(52) U.S. Cl. CPC A61K 31/519; A61K 31/4375; A61K 47/68037; A61K 47/6851; A61P 35/00; C07K 16/30

(57) ABSTRACT

This invention is in the area of improved combination treatments for a select group of hard-to-treat cancer patients, including, for example, those with advanced/metastatic triple negative breast cancer (TNBC), recurrent or metastatic urothelial cancer (mUC), or a Trop-2 overexpressing cancer such as non-small cell lung cancer (NSCLC), wherein the improved combination includes the use of trilaciclib and sacituzumab govitecan, and provides increased overall survival and/or reduced toxicity.

### CROSS-REFERENCE TO RELATED APPLICATIONS

This application is a continuation of International Application No. PCT/US2022/035995, filed Jul. 1, 2022, which claims the benefit of U.S. Provisional Application No. 63/217,716, filed Jul. 1, 2021. The entirety of each of these applications is hereby incorporated by reference herein for all purposes.

### FIELD OF THE INVENTION

This invention is in the area of improved combination treatments for a select group of hard-to-treat cancer patients, including, for example, those with advanced/metastatic triple negative breast cancer (TNBC), recurrent or metastatic urothelial cancer (mUC), or a Trop-2 overexpressing cancer including but not limited to non-small cell lung cancer (NSCLC), wherein the improved combination includes the use of trilaciclib and sacituzumab govitecan, and provides increased overall survival and/or reduced toxicity.

### BACKGROUND OF THE INVENTION

### Triple Negative Breast Cancer

Triple-negative breast cancer (TNBC) has been characterized by several aggressive clinicopathologic features, including onset at a younger age, large, high-grade tumors, and a propensity for visceral metastasis (Cheang et al. Basal-like breast cancer defined by five biomarkers has superior prognostic value than triple-negative phenotype. Clin Cancer Res. 2008;14(5):1368-76; Foulkes et al., Triple-negative breast cancer. N Engl J Med. 2010 Nov 11;363(20):1938-48.). The estimated median survival from the time of diagnosis is approximately 13-18 months and the median age at diagnosis is approximately 50 years (Kassam et al. Survival outcomes for patients with metastatic triple-negative breast cancer: implications for clinical practice and trial design. Clin Breast Cancer. 2009;9(1):29-33.; Yardley et al. nab-Paclitaxel plus carboplatin or gemcitabine versus gemcitabine plus carboplatin as first-line treatment of patients with triple-negative metastatic breast cancer: results from the tnAcity trial. Ann Oncol. 2018;29(8):1763-70.). Treatments which are effective for hormone receptor-positive breast cancer and human epidermal growth factor receptor 2 (HER2)-positive breast cancer, such as endocrine therapy or HER2-targeted therapies (e.g., trastuzumab) are not effective in TNBC, which lacks the expression of these markers; as such, chemotherapy remains the mainstay of treatment for TNBC. In particular, chemotherapies that target deoxyribonucleic acid (DNA) repair (e.g., platinum compounds) and cell proliferation (e.g., taxanes and anthracyclines, like doxorubicin) have been found to be the most effective in TNBC; however, these treatments are limited by toxicity and eventually all patients develop drug resistance.

In addition to the limitations described above, chemotherapy-induced immunosuppression may also affect anti-tumor efficacy due to an inability of the host immune system to effectively mount a response against the cancer. In 2019, accelerated approval was granted by the United States (US) Food and Drug Administration (FDA) and European Medicines Agency (EMA) for atezolizumab, a programmed death-ligand 1 (PD-L1) blocking antibody (immune checkpoint inhibitor [ICI]), in combination with nab-paclitaxel for patients with PD-L1 positive locally advanced unresectable/metastatic TNBC (Tecentriq® Package Insert, 2020). The FDA accelerated approval was based on the clinical trial results indicating improved progression-free survival (PFS) for patients with PD-L1 positive (PD-L1—stained tumor-infiltrating immune cells [IC] of any intensity covering >1% of the tumor area) tumors (median 7.4 months vs. 4.8 months; hazard ratio [HR]: 0.60 [0.48, 0.77]; p<0.0001). In addition, the PD-L1 positive subset lived for an average of 25 months when treated with atezolizumab plus nab-paclitaxel, compared with 18 months when given placebo plus nab-paclitaxel. The efficacy improvement observed with the addition of atezolizumab to nab-paclitaxel was associated with immune-related adverse events that occurred at relatively low frequencies, which could cause significant morbidity and mortality.

Unfortunately, following confirmatory studies, the approval of atezolizumab for use in PD-L1 positive subsets of patients with TNBC was subsequently withdrawn. Results published in Annal of Oncology in 2021 indicated that the trial failed to meet the primary end point of PFS superiority in the frontline treatment of patients with PD-L1 positivity (HR, 0.82; 95% CI, 0.60-1.12; P=0.20) (Miles et al. Primary results from IMpassion131, a double-blind, placebo-controlled, randomised phase III trial of first-line paclitaxel with or without atezolizumab for unresectable locally advanced/metastatic triple-negative breast cancer. Ann Oncol. 2021;32(8):994-1004. doi:10.1016/j.annonc.2021.05.801). Additionally, there was no difference in survival advantage in the PD-Ll—positive (HR 1.11, 95% CI 0.76-1.64) nor the intention to treat population.

Accelerated approval was also granted for pembrolizumab in combination with chemotherapy for the treatment of patients with locally recurrent unresectable or metastatic TNBC whose tumors express PD-L1 (CPS ≥10; CPS=combined positive score) as determined by a companion diagnostic FDA approved test (Keytruda® Package Insert, 2020). Approval was based on the main efficacy outcome measure of PFS in the subgroup of patients with CPS ≥10 (median PFS 9.7 months [95% confidence interval (CI): 7.6, 11.3] in the pembrolizumab plus chemotherapy arm and 5.6 months (95% CI:5.3, 7.5) in the placebo arm (HR 0.65; 95% CI: 0.49, 0.86; one-sided p-value=0.0012)

While the combination of certain ICIs with chemotherapy have indicated a meaningful step forward for the treatment of patients with PD-L1 positive locally advanced unresectable/metastatic TNBC, it should be noted that due to the potential treatment toxicities associated with ICIs, not all patients with PD-L1 positive TNBC are appropriate candidates for ICI treatment and, as would be expected, the patient population with PD-L1 negative TNBC may not derive benefit.

Additional available targeted therapies recently approved by the FDA are PARP inhibitors such as olaparib for the treatment of patients with germline BRCA-positive, HER2-negative metastatic breast cancer (MBC) who have previously received chemotherapy (approved in January 2018) and talazoparib for patients with deleterious or suspected deleterious germline BRCA-mutated, HER2-negative locally advanced or metastatic breast cancer (MBC) (approved in October 2018). While these two targeted therapies offer benefit to patients with TNBC, they are limited to those with germline BRCA mutations (˜9-18%, Hahnen et al., Germline Mutations in Triple-Negative Breast Cancer. Breast Care (Basel). 2017; 12(1): 15-19.).

Overall, patients with TNBC have few approved treatment options beyond standard chemotherapy. Even though there are now available targeted therapies for TNBC, these therapies are limited to those eligible patients expressing PD-L1-positive disease (ICIs) and/or those that carry a germline BRCA mutation (PARP inhibitors) and in the case of ICIs, come with additional toxicity. Novel therapeutic combinations that can offer similar or improved antitumor efficacy without the associated potential high-grade toxicities are clearly needed for all TNBC patients, regardless of PD-L1 or BRCA status.

### Metastatic Urothelial Carcinoma

Patients with metastatic urothelial carcinoma (mUC) with disease progression after combination platinum-based chemotherapy and immune checkpoint inhibitors (ICIs) also have limited treatment options (National Comprehensive Cancer Network: NCCN Clinical Practice Guidelines in Oncology: Bladder Cancer Version 5.2020. https://www.nccn.org/professionals/physician_gls/). Following progression, the only widely available agents indicated per NCCN and ESMO guidelines have been taxanes and vinflunine (approved in the European Union). These agents have response rates of approximately 10% with a median overall survival (OS) of 7-8 months (Petrylak et al: Ramucirumab plus docetaxel versus placebo plus docetaxel in patients with locally advanced or metastatic urothelial carcinoma after platinum-based therapy

(RANGE): A randomised, double-blind, phase 3 trial. Lancet 390:2266-2277, 2017; Raggi et al: Second-line single-agent versus doublet chemotherapy as salvage therapy for metastatic urothelial cancer: A systematic review and meta-analysis. Ann Oncol 27:49-61, 2016; Niegisch et al: A real-world data study to evaluate treatment patterns, clinical characteristics and survival outcomes for first- and second-line treatment in locally advanced and metastatic urothelial cancer patients in Germany. J Cancer 9:1337-1348, 2018; Fradet et al: Randomized phase III KEYNOTE-045 trial of pembrolizumab versus paclitaxel, docetaxel, or vinflunine in recurrent advanced urothelial cancer: Results of 2 years of follow-up. Ann Oncol 30:970-976, 2019; Di Lorenzo et al: Third-line chemotherapy for metastatic urothelial cancer: A retrospective observational study. Medicine (Baltimore) 94:e2297, 2015; Vlachostergios et al: Antibody-drug conjugates in bladder cancer. Bladder Cancer 4:247-259, 2018).

The therapeutic landscape for mUC in the United States has been expanded by the accelerated US Food and Drug Administration (FDA) approvals of erdafitinib, a pan-fibroblast growth factor receptor inhibitor for patients with tumors harboring FGFR2- or FGFR3-activating mutation or fusion (following platinum-based chemotherapy), and enfortumab vedotin (EV), a nectin-4-directed antibody-drug conjugate (ADC) following platinum-based chemotherapy and ICI (Padcev [Package Insert]. Northbrook, IL, Astellas Pharma US, 2019; FDA Grants Accelerated Approval to Enfortumab Vedotin-ejfv for Metastatic Urothelial Cancer [Press Release]. Silver Spring, MD: US Food and Drug Administration, Dec. 19, 2019; Loriot et al: Erdafitinib in locally advanced or metastatic urothelial carcinoma. N Engl J Med 381:338-348, 2019). Although both EV and erdafitinib have objective response rates (ORRs) of approximately 40%, most patients progress on these therapies. Moreover, erdafitinib is limited to patients with FGFR2/3 mutations or fusions (15%-20% of patients depending on cancer type) (de Almeida Carvalho et al: Estimation of percentage of patients with fibroblast growth factor receptor alterations eligible for off-label use of erdafitinib. JAMA Netw Open 2:e1916091, 2019).

Overall, patients with mUC currently have few treatment options beyond platinum-containing chemotherapy regimens. Even though there are now available targeted therapies for mUC, these therapies are limited to those eligible patients expressing PD-L1-positive disease (ICIs) which come with potential additional toxicity. Novel therapeutic combinations that can offer similar or improved antitumor efficacy without the associated high-grade toxicities are clearly needed for all mUC patients, regardless of PD-L1 status.

### Non-Small Cell Lung Cancer

More than more than 2 million cases of lung cancer and 1.75 million lung cancer related deaths were reported in 2018 (WHO, 2020). The American Cancer Society estimates that in 2020, ˜135,000 of those lung cancer deaths will occur in the US alone (American Cancer Society, 2020). Approximately 84% of these patients will be diagnosed with non-small cell lung cancer (NSCLC) and almost 70% will present with locally advanced or metastatic disease (ASCO, 2020; Little, 2007). Historically, treatment in the metastatic NSCLC setting has been driven almost exclusively by the use of systemic chemotherapy. However, in the last ten to twenty years, a greater understanding of the pathways that dictate tumor response and the advent of multiple targeted therapies has changed the landscape of treatment options significantly. Lung tumors are routinely tested for the presence of specific driver mutations (e.g., epidermal growth factor receptor (EGFR), anaplastic lymphoma kinase (ALK), BRAF, rearranged during translocation proto-oncogene (RET), AKT1, ERBB2, MEK1, MET, NRAS, PIK3CA, RET, TRK1, and c ROS oncogene 1 (ROS1), neurotrophic tyrosine receptor kinase (NTRK)), that predict a favorable response to targeted tyrosine kinase inhibitors (Kalemkerian, 2018). As many as 50% to 64% of patients have been identified as having a targetable genetic alteration and those that receive treatment have better outcomes (Kris, 2014; Barlesi, 2016).

For those patients without a targetable genetic alteration, treatment options have also changed considerably. Though systemic therapy remains an important component of treatment in this context, the advent of immunotherapy has significantly improved outcomes for this patient population. Multiple randomized trials, in both squamous and non-squamous histologies, have established that overall survival (OS) is improved with the addition of programmed cell death protein 1 (PD-1)/programmed death-ligand 1 (PD-L1) inhibitors (Spigel, 2019; Reck, 2016; Gandhi, 2018; Paz-Ares, 2018). Those with high levels of PD-L1 expression typically receive pembrolizumab or atezolizumab monotherapy in the first line followed by platinum-based chemotherapy in order to maximize treatment response and minimize toxicity. Those with a higher burden of disease requiring more aggressive initial treatment or with lower levels of PD L1 expression typically receive immunotherapy in combination with a platinum-based chemotherapy doublet.

Despite these improvements in the therapy for metastatic NSCLC, the majority of patients ultimately progress during or after treatment with immunotherapy and platinum-based chemotherapy. Management of this pretreated patient population is challenging and treatment options are limited to single agent chemotherapies such as docetaxel, pemetrexed (for those with non-squamous histology), and gemcitabine (Shepherd, 2000; Fossella, 2010; Gridelli, 2004; Hanna, 2004; Anderson, 2000).

Because of the limited treatment available to NSCLC patients who have advanced on immune-checkpoint inhibitors, novel therapies are needed following disease progression on currently clinically available treatments.

### Sacituzumab Govitecan

Recently, sacituzumab govitecan-hziy, has received U.S. FDA approval as a treatment in two very difficult to treat population: 1) patients with unresectable locally advanced or metastatic triple-negative breast cancer (mTNBC) who have received two or more prior systemic therapies, at least one of them for metastatic disease; and 2) locally advanced or metastatic urothelial cancer (mUC) who have previously received a platinum-containing chemotherapy and either programmed death receptor-1 (PD-1) or programmed death-ligand 1 (PD-L1) inhibitor. Sacituzumab govitecan is a Trop-2-directed humanized monoclonal antibody, hRS7 IgG1κ (also called sacituzumab), which binds to Trop-2 (the trophoblast cell-surface antigen-2) conjugated via a hydrolyzable linker (called CL2A) to the drug SN-38, a topoisomerase inhibitor. Trop-2 has been shown to be over-expressed in the majority of epithelial carcinomas, including breast, colon, prostate, pancreatic, urothelial, and lung cancers. Trop-2 plays an important role in anchorage-independent cell growth and tumorigenesis. Pharmacology data suggest that sacituzumab govitecan binds to Trop-2-expressing cancer cells and is internalized with the subsequent release of SN-38 via hydrolysis of the linker. SN-38, an active metabolite of irinotecan, interacts with topoisomerase I and prevents re-ligation of topoisomerase I-induced single strand breaks. The resulting DNA damage leads to apoptosis and cell death.

Initial clinical trials in patients with unresectable locally advanced or metastatic triple-negative breast cancer (mTNBC) who had relapsed after at least two prior chemotherapies for breast cancer (one of which could be in the neoadjuvant or adjuvant setting provided progression occurred within a 12 month period) showed a median progression free survival of 4.8 months (compared to 1.7 months on single agent chemotherapy) and an overall survival of 11.8 months (compared to 6.9 months on single agent chemotherapy). Initial clinical trials in patients with locally advanced or mUC who have received prior treatment with a platinum-containing chemotherapy, e.g., cisplatin, carboplatin, or oxaliplatin, and either PD-1 or PD-L1 inhibitor provided an overall response rate of 27.7%, with a median response duration of 7.1 months.

Despite these promising results, the administration of sacituzumab govitecan is associated with significant and deleterious side effects, and its U.S. FDA approved label contains a “black box warning” relating to the development of severe, life threatening, or fatal neutropenia and severe diarrhea. As reported in the U.S. FDA approved label for sacituzumab govitecan-hziy (Trodelvy®), neutropenia occurred in 61% of patients treated with sacituzumab govitecan-hziy. Grade 3-4 neutropenia occurred in 47% of patients. Febrile neutropenia occurred in 7% of patients. Diarrhea occurred in 65% of all patients treated with Trodelvy®. Grade 3-4 diarrhea occurred in 12% of all patients treated with Trodelvy 0. One patient had intestinal perforation following diarrhea. Neutropenic colitis occurred in 0.5% of patients. Other side effects associated with the use of sacituzumab govitecan-hziy include fatigue, alopecia, acute kidney injury, infection, anemia, and thrombocytopenia.

Importantly, these side effects, in addition to being life threatening or fatal, can also be dose-limiting or result in the discontinuation of treatment. For example, sacituzumab govitecan was permanently discontinued in mTNBC patients enrolled in the ASCENT study for adverse reactions in 5% of patients. Forty-five percent (45%) of patients in the ASCENT study experienced an adverse reaction leading to treatment interruption, and 22% of patients experienced an adverse reaction leading to a dose reduction. The discontinuation of, or dose reduction of, sacituzumab govitecan may result in decreased efficacy and contribute to the development of disease resistance and advancement. Granulocyte-colony stimulating factor (G-CSF) was used as a rescue therapy in 44% of patients in the ASCENT study.

Because of the already limited treatments available to TNBC and mUC patients who have advanced despite standard of care and newly approved therapies, the toxicity of sacituzumab govitecan presents significant hurdles to its consistent and long-term use as a therapeutic.

### SUMMARY OF THE INVENTION

The present invention provides improved methods for treating human patients with advanced and/or metastatic triple negative breast cancer (TNBC) or alternatively, recurrent or metastatic urothelial cancer (mUC), or still alternatively a Trop-2 overexpressing cancer such as NSCLC in specific, select patient subgroups by administering the short acting, selective, and reversible cyclin dependent kinase (CDK) 4/6 inhibitor trilaciclib, or a pharmaceutically acceptable salt thereof, in a specifically timed therapeutic protocol with sacituzumab govitecan (also known as sacituzumab govitecan-hziy; Trodelvy®), or a biosimilar thereof, wherein trilaciclib is administered less than 24 hours prior to, for example, about 4 hours or less prior to, the administration of sacituzumab govitecan. The inclusion of the selective CDK4/6 inhibitor trilaciclib—also known as Cosela®—in a sacituzumab govitecan therapeutic regimen may provide for improved survival outcomes, including overall survival (OS) and/or progression free survival (PFS). Importantly, the inclusion of trilaciclib in a sacituzumab govitecan therapeutic regimen provides significantly reduced toxicity for these difficult to treat patients compared to the administration of sacituzumab govitecan alone, including a reduction or prevention of chemotherapy-induced myelosuppression (CIM), a reduction or prevention of chemotherapy-induced neutropenia, a reduction or prevention of chemotherapy-induced anemia, a reduction or prevention of chemotherapy-induced mucositis, a reduction or prevention of chemotherapy-induced diarrhea, a reduction or prevention of chemotherapy-induced stomatitis, a reduction or prevention of chemotherapy-induced gastrointestinal disorders, and/or a reduction or prevention of alopecia

Without wishing to be bound by any single theory, it is believed that trilaciclib administered before sacituzumab govitecan protects the bone marrow from the cytotoxic effects of sacituzumab govitecan, while also inducing anti-tumor immune-enhancing effects by differentially arresting CD8+ T cell and Treg subsets, followed by a faster recovery of CD8+ T cells than Tregs in the tumor microenvironment. These mechanisms result in both improved safety—through the significant reduction of side effects—and anti-tumor activity. In addition, it is further believed that trilaciclib administered before sacituzumab govitecan protects other CDK4/6 replication dependent heathy cells, for example epithelial cells of the gastrointestinal tract. By protecting these cells from the significant side-effects of sacituzumab govitecan, a reduction in dosing delays, a reduction in dosage reductions, and a reduction in treatment discontinuation in this difficult to treat population can be realized, and the use of sacituzumab govitecan expanded. Furthermore, the use of trilaciclib in combination with sacituzumab govitecan may result in a reduction in the use of rescue therapies, for example, granulocyte-colony stimulation factor (G-CSF), granulocyte-macrophage colony stimulating factor (GM-CSF), or erythropoiesis-stimulating agent. Thus, administering trilaciclib prior to sacituzumab govitecan can improve antitumor efficacy while minimizing myelotoxicity in patients with Trop-2 expressing tumors.

Early results from initial human clinical trials (see, e.g., NCT05113966) have shown trilaciclib's ability to drastically reduce side effects associated with sacituzumab govitecan administration, including a reduction in the incidence of severe (Grade 3/4) neutropenia (see, for example, Example 1, FIG. 2A). In the initial studies, 8 female patients (median age 55.0 years) had been enrolled and completed a median 3 (range 1-6) 21-day cycles, wherein sacituzumab govitecan was administered on days 1 and 8 of each 21-day cycle, and trilaciclib was administered over a 30 minute intravenous administration within 4 hours prior to administration of sacituzumab govitecan on days 1 and 8 of each 21-day cycle (see, e.g., FIG. 1). All patients had received a prior taxane and 7 had received a prior immune checkpoint inhibitor (5 atezolizumab, 2 pembrolizumab; 4 in the adjuvant setting, 3 in the metastatic setting). Severe (Grade 3/4) neutropenia occurred in only 1 patient (12.5% with trilaciclib compared with 52% in the ASCENT study). No patients had Grade 3/4 anemia or thrombocytopenia, and none had febrile neutropenia or a serious infection. Granulocyte colony-stimulating factors were administered to 2 patients (25%) and 1 patient required intravenous antibiotics. No patients required red blood cell/platelet transfusions or erythropoiesis-stimulating agents. No patients were withdrawn due to an adverse event and no serious adverse events have been reported to date. There was only 1 treatment-related adverse event of diarrhea. Thus, the initial studies indicate that the inclusion of trilaciclib drastically reduces the side effects associated with sacituzumab govitecan.

### Metastatic/Locally Advanced Triple Negative Breast Cancer

In one aspect, provided herein is an improved method of treating metastatic and/or advanced triple negative breast cancer comprising administering to a human patient with metastatic or locally advanced triple negative breast cancer trilaciclib, or a pharmaceutically acceptable salt thereof, in combination with sacituzumab govitecan, wherein trilaciclib is administered prior to administration of sacituzumab govitecan, for example about 24 hours, 18 hours, 16 hours, 12 hours, 8 hours, 6 hours, 4 hours, 2 hours, 1 hour, or 30 minutes prior to administration of sacituzumab govitecan. In some embodiments, the patient has previously received at least 2 prior chemotherapeutic treatments, at least 1 in the metastatic setting.

In some embodiments, the method comprises administering to a patient having TNBC a 21-day chemotherapeutic treatment cycle, an effective amount of trilaciclib on day 1 and day 8, and administering an effective amount of sacituzumab govitecan on day 1 and day 8, wherein the trilaciclib is administered prior to the administration of sacituzumab govitecan. In some embodiments, trilaciclib is administered 18 hours, 16 hours, 12 hours, 8 hours, 6 hours, 4 hours, 2 hours, 1 hour, or 30 minutes prior to administration of sacituzumab govitecan. In some embodiments, trilaciclib is administered about 4 hours or less prior to administration of sacituzumab govitecan. In some embodiments, the 21-day therapeutic treatment cycle is repeated at least 2 times, at least 4 times, at least 6 times, at least 8 times, at least 10 times, at least 12 times, at least 14 times, at least 16 times, at least 18 times, at least 20 times, at least 22 times, at least 24 times, at least 26 times, at least 28 times, at least 30 times, at least 32 times, at least 34 times, or more than 34 times. In some embodiments, the 21-day therapeutic agent is repeated up to 34 times. In some embodiments, the 21-day therapeutic treatment cycle is repeated or continuous until disease progression.

In some embodiments, the method comprises administering to a patient having TNBC a 21-day chemotherapeutic treatment cycle, an effective amount of trilaciclib on day 1, day 8, and day 15, and administering an effective amount of sacituzumab govitecan on day 1 and day 8, wherein the trilaciclib is administered on day 1 and day 8 prior to the administration of sacituzumab govitecan. In some embodiments, trilaciclib is administered 18 hours, 16 hours, 12 hours, 8 hours, 6 hours, 4 hours, 2 hours, 1 hour, or 30 minutes prior to administration of sacituzumab govitecan on day 1 and day 8. In some embodiments, trilaciclib is administered about 4 hours or less prior to administration of sacituzumab govitecan. In some embodiments, the 21-day therapeutic treatment cycle is repeated at least 2 times, at least 4 times, at least 6 times, at least 8 times, at least 10 times, at least 12 times, at least 14 times, at least 16 times, at least 18 times, at least 20 times, at least 22 times, at least 24 times, at least 26 times, at least 28 times, at least 30 times, at least 32 times, at least 34 times, or more than 34 times. In some embodiments, the 21-day therapeutic agent is repeated up to 34 times. In some embodiments, the 21-day therapeutic treatment cycle is repeated or continuous until disease progression.

In some embodiments, the method comprises administering to a patient having advanced/metastatic TNBC in a third-line setting during a 21-day chemotherapeutic treatment cycle, an effective amount of trilaciclib on day 1 and day 8, and administering an effective amount of sacituzumab govitecan on day 1 and day 8, wherein the trilaciclib is administered prior to the administration of sacituzumab govitecan. In some embodiments, trilaciclib is administered 18 hours, 16 hours, 12 hours, 8 hours, 6 hours, 4 hours, 2 hours, 1 hour, or 30 minutes prior to administration of sacituzumab govitecan. In some embodiments, trilaciclib is administered about 4 hours or less prior to administration of sacituzumab govitecan. In some embodiments, the 21-day therapeutic treatment cycle is repeated at least 2 times, at least 4 times, at least 6 times, at least 8 times, at least 10 times, at least 12 times, at least 14 times, at least 16 times, at least 18 times, at least 20 times, at least 22 times, at least 24 times, at least 26 times, at least 28 times, at least 30 times, at least 32 times, at least 34 times, or more than 34 times. In some embodiments, the 21-day therapeutic agent is repeated up to 34 times. In some embodiments, the 21-day therapeutic treatment cycle is repeated or continuous until disease progression.

In some embodiments, the method comprises administering to a patient having advanced/metastatic TNBC in a third-line setting during a 21-day chemotherapeutic treatment cycle, an effective amount of trilaciclib on day 1, day 8, and day 15, and administering an effective amount of sacituzumab govitecan on day 1 and day 8, wherein the trilaciclib is administered on day 1 and day 8 prior to the administration of sacituzumab govitecan. In some embodiments, trilaciclib is administered 18 hours, 16 hours, 12 hours, 8 hours, 6 hours, 4 hours, 2 hours, 1 hour, or 30 minutes prior to administration of sacituzumab govitecan on day 1 and day 8. In some embodiments, trilaciclib is administered about 4 hours or less prior to administration of sacituzumab govitecan. In some embodiments, the 21-day therapeutic treatment cycle is repeated at least 2 times, at least 4 times, at least 6 times, at least 8 times, at least 10 times, at least 12 times, at least 14 times, at least 16 times, at least 18 times, at least 20 times, at least 22 times, at least 24 times, at least 26 times, at least 28 times, at least 30 times, at least 32 times, at least 34 times, or more than 34 times. In some embodiments, the 21-day therapeutic agent is repeated up to 34 times. In some embodiments, the 21-day therapeutic treatment cycle is repeated or continuous until disease progression.

In some embodiments, prior to the administration of sacituzumab govitecan, the patient is premedicated for the prevention of infusion reactions and chemotherapy-induced nausea and vomiting (CINV). In some embodiments, prior to the administration of sacituzumab govitecan, the patient is premedicated with antipyretics, histamine receptor 1 (H1) and histamine receptor 2 (H2) blockers, and corticosteroids for the prevention of infusion reactions. In some embodiments, prior to the administration of sacituzumab govitecan, the patient is premedicated with dexamethasone in combination with either a serotonin 5-HT3 receptor antagonist or a Neurokinin 1 (NK1) receptor antagonist for the prevention of chemotherapy-induced nausea and vomiting (CINV).

In some embodiments, the patient being treated the sacituzumab govitecan/trilaciclib administration protocol has a TNBC that is CDK4/6-positive. In some embodiments, the TNBC to be treated is CDK4/6-negative. In some embodiments, the TNBC to be treated has at least one of the following characteristics:
a. a CCNE1 amplification;
b. a CCNE2 amplification; or
c. a Retinoblastoma protein 1 (Rb1) loss, which is defined as i) homozygous deletion, ii) a frameshift mutation, or iii) a stop-gained mutation (i.e., a mutation that results in a premature termination codon (a stop codon was gained. In still other alternative embodiments, the TNBC is CDK4/6 indeterminate. In some embodiments, the patient being treated is being treated in a third-line setting.

In some embodiments, the patient being treated with the trilaciclib/sacituzumab govitecan protocol has a documented PD-L1 status positive TNBC. In some embodiments, the patient being treated has a documented PD-L1 status positive PD-L1 staining tumor-infiltrating immune cells or tumor cells as confirmed by an in vitro diagnostic (IVD) assay, for example, the Ventana SP-142 assay or I IHC 22C3 pharmDx PDL1 assay or other approved assay.

In some embodiments, the patient being treated in the trilaciclib/sacituzumab govitecan chemotherapeutic protocol described herein has documented disease progression during or after 2 prior lines of systemic treatment for TNBC. In some embodiments, one prior line of treatment either in the neoadjuvant, adjuvant, or advanced/metastatic setting is with a taxane therapy. In some embodiments, the taxane therapy is paclitaxel. In some embodiments, the taxane therapy is docetaxel. In some embodiments, one prior line of treatment either in the neoadjuvant, adjuvant, or advanced/metastatic setting is with an anthracycline therapy. In some embodiments the anthracycline therapy is doxorubicin. In some embodiments the anthracycline therapy is daunorubicin. In some embodiments the anthracycline therapy is idarubicin. In some embodiments the anthracycline therapy is epirubicin. In some embodiments the anthracycline therapy is mitoxantrone.

In some embodiments, one prior line of treatment either in the neoadjuvant, adjuvant or advanced/metastatic setting received prior to administration of the trilaciclib/sacituzumab govitecan is with a platinum-based chemotherapy including, but not limited to, cisplatin, carboplatin, oxaliplatin, nedaplatin, satraplatin, platinum, and lobaplatin. In some embodiments, the platinum-based chemotherapy is carboplatin. In some embodiments, the platinum-based chemotherapy is cisplatin. In some embodiments, one prior line of treatment either in the neoadjuvant, adjuvant, or advanced/metastatic setting is with an antimetabolite chemotherapy. In some embodiments, the antimetabolite chemotherapy is gemcitabine. In some embodiments, the antimetabolite chemotherapy is capecitabine. In some embodiments, one prior line of treatment either in the neoadjuvant, adjuvant or advanced/metastatic setting is with a microtubule inhibitor. In some embodiments, the microtubule inhibitor is eribulin. In some embodiments, the microtubule inhibitor is vinorelbine. In some embodiments, the microtubule inhibitor is ixabepilone. In some embodiments, one prior line of treatment either in the neoadjuvant, adjuvant or advanced/metastatic setting is with an alkylating agent. In some embodiments, the alkylating agent is cyclophosphamide. In some embodiments, one prior line of treatment either in the neoadjuvant, adjuvant or advanced/metastatic setting for patients with documented germline BRCAl/BRCA2 mutation is a poly-ADP-ribose polymerase (PARP) inhibitor. In some embodiments, the PARP inhibitor is olaparib. In some embodiments, the PARP inhibitor is talazoparib.

In some embodiments, one prior line of treatment either in the neoadjuvant, adjuvant or advanced/metastatic setting for patients with a positive PD1 or PDL1 status is a PD-1 or PDL1 inhibitor. In some embodiments, the PD-1 inhibitor is pembrolizumab. In some embodiments, the PD-1 inhibitor is nivolumab. In some embodiments, the PD-1 inhibitor is cemiplimab. In some embodiments, the PD-1 inhibitor is CS1003. In some embodiments, the PD-1 inhibitor is tislelizumab. In some embodiments, the PD-1 inhibitor is dostarlimab. In some embodiments, the PD-1 inhibitor is JTX-4014. In some embodiments, the PD-1 inhibitor is spartalizumab. In some embodiments, the PD-1 inhibitor is camrelizumab. In some embodiments, the PD-1 inhibitor is sintilimab. In some embodiments, the PD-1 inhibitor is toripalimab. In some embodiments, the PD-1 inhibitor is retifanlimab. In some embodiments, the PD-1 inhibitor is AMP-224. In some embodiments, the PD-1 inhibitor is AMP-514. In some embodiments, the PD-1 inhibitor is pidilizumab. In some embodiments, the PD-1 inhibitor is sasanlimab. In some embodiments, the PD-1 inhibitor is zimberelimab. In some embodiments, the PD-L1 inhibitor is atezolizumab. In some embodiments, the PD-L1 inhibitor is avelumab. In some embodiments, the PD-L1 inhibitor is durvalumab. In some embodiments, the PD-L1 inhibitor is sugemalimab. In some embodiments, the PD-L1 inhibitor is envafolimab. In some embodiments, the PD-L1 inhibitor is cosibelimab. In some embodiments, the PD-L1 inhibitor is AUNP12. In some embodiments, the PD-L1 inhibitor is CA-170. In some embodiments, the PD-L1 inhibitor is BMS-986189. In some embodiments, the PD-L1 inhibitor is BMS-936559. In some embodiments, the PD-L1 inhibitor is lodapolimab. In some embodiments, the PD-L1 inhibitor is adebrelimab. In some embodiments, the PD-L1 inhibitor is CBT-502. In some embodiments, the PD-L1 inhibitor is BGB-A33.

Metastatic Urothelial Cancer (mUC)

In an alternative aspect, the improved method comprises administering to a patient with mUC trilaciclib, or a pharmaceutically acceptable salt thereof, in combination with sacituzumab govitecan, wherein trilaciclib is administered prior to administration of sacituzumab govitecan, for example about 24 hours, 18 hours, 16 hours, 12 hours, 8 hours, 6 hours, 4 hours, 2 hours, 1 hour, or 30 minutes prior to administration of sacituzumab govitecan. In some embodiments, the trilaciclib/sancituzumab govitecan combination is administered to the patient in a second-line or third-line treatment setting for advanced/metastatic urothelial cancer who have prior exposure to 1) a platinum-containing chemotherapy in the recurrent or metastatic setting and 2) an immune checkpoint inhibitor, for example, a PD-1 or PD-L1 inhibitor, either as monotherapy in the first-line treatment setting or in combination with a first line chemotherapeutic regimen, and who have developed disease progression.

In particular embodiments, the method comprises administering to the patient in a 21-day trilaciclib/sacituzumab govitecan treatment cycle, an effective amount of trilaciclib on day 1 and day 8, and an effective amount of sacituzumab govitecan-hziy on day 1 and day 8, wherein the trilaciclib is administered less than about four hours prior to the administration of sacituzumab govitecan. In some embodiments, trilaciclib is administered 18 hours, 16 hours, 12 hours, 8 hours, 6 hours, 4 hours, 2 hours, 1 hour, or 30 minutes prior to administration of sacituzumab govitecan. In some embodiments, trilaciclib is administered about 4 hours or less prior to administration of sacituzumab govitecan. In some embodiments, the 21-day therapeutic treatment cycle is repeated at least 2 times, at least 4 times, at least 6 times, at least 8 times, at least 10 times, at least 12 times, at least 14 times, at least 16 times, at least 18 times, at least 20 times, at least 22 times, at least 24 times, at least 26 times, at least 28 times, at least 30 times, at least 32 times, at least 34 times, or more than 34 times. In some embodiments, the 21-day trilaciclib/sacituzumab govitecan treatment cycle is repeated up to 34 times. In some embodiments, the 21-day trilaciclib/sacituzumab govitecan treatment cycle is repeated continuously. In some embodiments, the 21-day trilaciclib/sacituzumab govitecan treatment cycle is repeated until disease progression.

In alternative embodiments, the method comprises administering to the patient in a 21-day trilaciclib/sacituzumab govitecan treatment cycle, an effective amount of trilaciclib on day 1, day 8, and day 15, and an effective amount of sacituzumab govitecan-hziy on day 1 and day 8, wherein the trilaciclib is administered less than about four hours prior to the administration of sacituzumab govitecan on day 1 and day 8. In some embodiments, trilaciclib is administered 18 hours, 16 hours, 12 hours, 8 hours, 6 hours, 4 hours, 2 hours, 1 hour, or 30 minutes prior to administration of sacituzumab govitecan. In some embodiments, trilaciclib is administered about 4 hours or less prior to administration of sacituzumab govitecan. In some embodiments, the 21-day therapeutic treatment cycle is repeated at least 2 times, at least 4 times, at least 6 times, at least 8 times, at least 10 times, at least 12 times, at least 14 times, at least 16 times, at least 18 times, at least 20 times, at least 22 times, at least 24 times, at least 26 times, at least 28 times, at least 30 times, at least 32 times, at least 34 times, or more than 34 times. In some embodiments, the 21-day trilaciclib/sacituzumab govitecan treatment cycle is repeated up to 34 times. In some embodiments, the 21-day trilaciclib/sacituzumab govitecan treatment cycle is repeated continuously. In some embodiments, the 21-day trilaciclib/sacituzumab govitecan treatment cycle is repeated until disease progression.

In some embodiments, prior to the administration of trilaciclib/sacituzumab govitecan, the patient is premedicated for the prevention of infusion reactions and chemotherapy-induced nausea and vomiting (CINV). In some embodiments, prior to the administration of trilaciclib/sacituzumab govitecan, the patient is premedicated with antipyretics, histamine receptor 1 (H1) and histamine receptor 2 (H2) blockers, and corticosteroids for the prevention of infusion reactions. In some embodiments, prior to the administration of trilaciclib/sacituzumab govitecan, the patient is premedicated with dexamethasone in combination with either a serotonin 5-HT3 receptor antagonist or a Neurokinin 1 (NK1) receptor antagonist for the prevention of chemotherapy—induced nausea and vomiting (CINV).

In some embodiments, the patient has a mUC that is CDK4/6-positive. In alternative embodiments, the mUC to be treated is CDK4/6-negative. In some embodiments, the mUC to be treated has at least one of the following characteristics:
a. a CCNE1 amplification;
b. a CCNE2 amplification; or
c. a Retinoblastoma protein 1 (Rb1) loss, which is defined as i) homozygous deletion, ii) a frameshift mutation, or iii) a stop-gained mutation (i.e., a mutation that results in a premature termination codon (a stop codon was gained)). In alternative embodiments, the mUC to be treated is CDK4/6-positive. In still other alternative embodiments, the mUC is CDK4/6 indeterminate.

In some embodiments, the mUC patient being treated in the second-line or third-line trilaciclib/sacituzumab govitecan protocol has a documented PD-L1 status positive mUC. In some embodiments, the patient being treated in the second-line or third-line trilaciclib/sacituzumab govitecan protocol has a documented PD-L1 status positive mUC of >10% PD-L1 staining tumor-infiltrating immune cells or >50% of tumor cells as confirmed by an in vitro diagnostic (IVD) assay, for example, the Ventana SP-142 assay or I IHC 22C3 pharmDx PDL1 assay or other approved assay. In some embodiments, the patient being treated in the second-line or third-line trilaciclib/sacituzumab govitecan administration protocol has a documented PD-L1 status positive mUC with >20% PD-L1 staining of tumor cells as confirmed by an in vitro diagnostic (IVD) assay, for example, the Ventana SP-142 assay or I IHC 22C3 pharmDx PDL1 assay. In an alternative embodiment, the patient being treated in the second-line or third-line trilaciclib/sacituzumab govitecan chemotherapeutic protocol has a documented PD-L1 status positive mUC with ≥1% PD-L1 staining of tumor cells as determined by an FDA-approved test. In an alternative embodiment, the patient being treated in the second-line or third-line antibody drug conjugate chemotherapeutic protocol has a documented PD-L1 status negative mUC. In an alternative embodiment, the patient being treated in the second-line or third-line antibody drug conjugate chemotherapeutic protocol has a documented PD-L1 status negative mUC with ≤1% PD-L1 staining of tumor cells as determined by an FDA-approved test.

In some embodiments, the patient with mUC being treated in the second-line or third-line trilaciclib/sacituzumab govitecan administration protocol has previously been treated with a platinum-containing chemotherapy protocol and an ICI inhibitor. In some embodiments, the platinum-containing chemotherapy is cisplatin and gemcitabine. In some embodiments, the platinum-containing chemotherapy is cisplatin, methotrexate, vinblastine and doxorubicin. In some embodiments, the platinum-containing chemotherapy is cisplatin, gemcitabine and paclitaxel. In some embodiments, the platinum-containing chemotherapy is carboplatin and gemcitabine. In some embodiments, the platinum-containing chemotherapy is carboplatin, methotrexate, vinblastine and doxorubicin. In some embodiments, the ICI is a PD-1 inhibitor. In some embodiments, the PD-1 inhibitor is nivolumab. In some embodiments, the PD-1 inhibitor is cemiplimab. In some embodiments, the PD-1 inhibitor is CS1003. In some embodiments, the PD-1 inhibitor is tislelizumab. In some embodiments, the PD-1 inhibitor is dostarlimab. In some embodiments, the PD-1 inhibitor is JTX-4014. In some embodiments, the PD-1 inhibitor is spartalizumab. In some embodiments, the PD-1 inhibitor is camrelizumab. In some embodiments, the PD-1 inhibitor is sintilimab. In some embodiments, the PD-1 inhibitor is toripalimab. In some embodiments, the PD-1 inhibitor is retifanlimab. In some embodiments, the PD-1 inhibitor is AMP-224. In some embodiments, the PD-1 inhibitor is AMP-514. In some embodiments, the PD-1 inhibitor is pidilizumab. In some embodiments, the PD-1 inhibitor is sasanlimab. In some embodiments, the PD-1 inhibitor is zimberelimab.

In some embodiments, the PD-L1 inhibitor is atezolizumab. In some embodiments, the PD-L1 inhibitor is avelumab. In some embodiments, the PD-L1 inhibitor is durvalumab. In some embodiments, the PD-L1 inhibitor is sugemalimab. In some embodiments, the PD-L1 inhibitor is envafolimab. In some embodiments, the PD-L1 inhibitor is cosibelimab. In some embodiments, the PD-L1 inhibitor is AUNP12. In some embodiments, the PD-L1 inhibitor is CA-170. In some embodiments, the PD-L1 inhibitor is BMS-986189. In some embodiments, the PD-L1 inhibitor is BMS-936559. In some embodiments, the PD-L1 inhibitor is lodapolimab. In some embodiments, the PD-L1 inhibitor is adebrelimab. In some embodiments, the PD-L1 inhibitor is CBT-502. In some embodiments, the PD-L1 inhibitor is BGB-A33.

### Additional Trop-2 Overexpressing Targeted Cancers

In an alternative aspect, provided herein is an improved method comprising administering trilaciclib, or a pharmaceutically acceptable salt thereof, in combination with sacituzumab govitecan, wherein trilaciclib is administered prior to administration of sacituzumab govitecan, for example about 24 hours, 18 hours, 16 hours, 12 hours, 8 hours, 6 hours, 4 hours, 2 hours, 1 hour, or 30 minutes prior to administration of sacituzumab govitecan, to a patient having an advanced/metastatic cancer that overexpress Trop-2, wherein the Trop-2 overexpressing cancer is selected from the group consisting of breast cancer, cervical cancer, colon or colorectal cancer, endometroid endometrial cancer, esophageal cancer, gastric cancer, a glioma, hilar cholangiocarcinoma, squamous cell carcinoma of the oral cavity, gastrointestinal cancer, chronic lymphocytic lymphoma, extranodal NK/T-cell lymphoma, non-Hodgkin's lymphoma, Raj i Burkitt lymphoma, small-sized pulmonary adenocarcinoma, ovarian cancer, pancreatic cancer, prostate cancer, stomach carcinoma, thyroid carcinoma, uterine cancer, and lung cancer, including small cell lung cancer and non-small cell lung cancer. In some embodiments, the patient has endometrial cancer. In some embodiments, the patient has bladder cancer. In some embodiments, the patient has prostate cancer. In some embodiments, the patient has HR+/HER2− Metastatic Breast Cancer.

In some embodiments, the Trop-2 overexpressing cancer is non-small cell lung cancer (NSCLC). In a particular embodiment, the NSCLC is metastatic or advanced NSCLC. In some embodiments, the NSCLC has progressed on or after platinum-based chemotherapy and PD-1 or PD-L1 inhibitor therapy received either in combination or sequentially.

In some embodiments, the method comprises administering to the patient having a cancer which overexpresses Trop-2 in a 21-day trilaciclib/sacituzumab govitecan treatment cycle, an effective amount of trilaciclib on day 1 and day 8, and an effective amount of sacituzumab govitecan on day 1 and day 8, wherein the trilaciclib is administered less than about four hours prior to the administration of sacituzumab govitecan. In some embodiments, trilaciclib is administered 18 hours, 16 hours, 12 hours, 8 hours, 6 hours, 4 hours, 2 hours, 1 hour, or 30 minutes prior to administration of sacituzumab govitecan. In some embodiments, trilaciclib is administered about 4 hours or less prior to administration of sacituzumab govitecan. In some embodiments, the 21-day therapeutic treatment cycle is repeated at least 2 times, at least 4 times, at least 6 times, at least 8 times, at least 10 times, at least 12 times, at least 14 times, at least 16 times, at least 18 times, at least 20 times, at least 22 times, at least 24 times, at least 26 times, at least 28 times, at least 30 times, at least 32 times, at least 34 times, or more than 34 times. In some embodiments, the 21-day therapeutic agent is repeated up to 34 times. In some embodiments, the 21-day trilaciclib/sacituzumab govitecan treatment cycle is repeated continuously. In some embodiments, the 21-day trilaciclib/sacituzumab govitecan treatment cycle is repeated until disease progression. In some embodiments, the Trop-2 overexpressing cancer is non-small cell lung cancer (NSCLC). In a particular embodiment, the NSCLC is metastatic or advanced NSCLC. In some embodiments, the NSCLC has progressed on or after platinum-based chemotherapy and PD-1 or PD-L1 inhibitor therapy received either in combination or sequentially.

In alternative embodiments, the method comprises administering to the patient having a cancer which overexpresses Trop-2 in a 21-day trilaciclib/sacituzumab govitecan treatment cycle, an effective amount of trilaciclib on day 1, day 8, and day 15, and an effective amount of sacituzumab govitecan on day 1 and day 8, wherein the trilaciclib is administered less than about four hours prior to the administration of sacituzumab govitecan. In some embodiments, trilaciclib is administered 18 hours, 16 hours, 12 hours, 8 hours, 6 hours, 4 hours, 2 hours, 1 hour, or 30 minutes prior to administration of sacituzumab govitecan. In some embodiments, trilaciclib is administered about 4 hours or less prior to administration of sacituzumab govitecan on day 1 and day 8. In some embodiments, the 21-day therapeutic treatment cycle is repeated at least 2 times, at least 4 times, at least 6 times, at least 8 times, at least 10 times, at least 12 times, at least 14 times, at least 16 times, at least 18 times, at least 20 times, at least 22 times, at least 24 times, at least 26 times, at least 28 times, at least 30 times, at least 32 times, at least 34 times, or more than 34 times. In some embodiments, the 21-day therapeutic agent is repeated up to 34 times. In some embodiments, the 21-day trilaciclib/sacituzumab govitecan treatment cycle is repeated continuously. In some embodiments, the 21-day trilaciclib/sacituzumab govitecan treatment cycle is repeated until disease progression. In some embodiments, the Trop-2 overexpressing cancer is non-small cell lung cancer (NSCLC). In a particular embodiment, the NSCLC is metastatic or advanced NSCLC. In some embodiments, the NSCLC has progressed on or after platinum-based chemotherapy and PD-1 or PD-L1 inhibitor therapy received either in combination or sequentially.

In some embodiments, the patient has a Trop-2 overexpressing cancer that is CDK4/6-positive. In alternative embodiments, the Trop-2 overexpressing cancer to be treated is CDK4/6-negative. In some embodiments, the Trop-2 overexpressing cancer to be treated has at least one of the following characteristics:
a. a CCNE1 amplification;
b. a CCNE2 amplification; or
c. a Retinoblastoma protein 1 (Rb1) loss, which is defined as i) homozygous deletion, ii) a frameshift mutation, or iii) a stop-gained mutation (i.e., a mutation that results in a premature termination codon (a stop codon was gained)). In alternative embodiments, the Trop-2 overexpressing cancer to be treated is CDK4/6-positive. In still other alternative embodiments, the Trop-2 overexpressing cancer is CDK4/6 indeterminate.

In some embodiments, the Trop-2 overexpressing cancer patient being treated with the trilaciclib/sacituzumab govitecan protocol has a documented PD-L1 status positive Trop-2 overexpressing cancer. In some embodiments, the patient being treated has a documented PD-L1 status positive PD-L1 staining tumor-infiltrating immune cells or tumor cells as confirmed by an in vitro diagnostic (IVD) assay, for example, the Ventana SP-142 assay or I IHC 22C3 pharmDx

PDLL assay or other approved assay.

In some embodiments, the methods described above for the treatment of TNBC, mUC, and/or a Trop-2 expressing cancer further comprise the administration of an immune-checkpoint inhibitor. In some embodiments, the immune checkpoint inhibitor is selected from a PD-1 inhibitor, a PD-L1 inhibitor, a CTLA-4 inhibitor, a TIM3 inhibitor, a TIGIT inhibitor, a LAG3 inhibitor, a VISTA inhibitor, or a SIGLEC7 inhibitor. In some embodiments, the immune checkpoint inhibitor is a PD-1 inhibitor or PD-L1 inhibitor. In some embodiments, the PD-1 inhibitor is nivolumab. In some embodiments, the PD-1 inhibitor is cemiplimab. In some embodiments, the PD-1 inhibitor is CS1003. In some embodiments, the PD-1 inhibitor is tislelizumab. In some embodiments, the PD-1 inhibitor is dostarlimab. In some embodiments, the PD-1 inhibitor is JTX-4014. In some embodiments, the PD-1 inhibitor is spartalizumab. In some embodiments, the PD-1 inhibitor is camrelizumab. In some embodiments, the PD-1 inhibitor is sintilimab. In some embodiments, the PD-1 inhibitor is toripalimab. In some embodiments, the PD-1 inhibitor is retifanlimab. In some embodiments, the PD-1 inhibitor is AMP-224. In some embodiments, the PD-1 inhibitor is AMP-514. In some embodiments, the PD-1 inhibitor is pidilizumab. In some embodiments, the PD-1 inhibitor is sasanlimab. In some embodiments, the PD-1 inhibitor is zimberelimab. In some embodiments, the PD-L1 inhibitor is atezolizumab. In some embodiments, the PD-L1 inhibitor is avelumab. In some embodiments, the PD-L1 inhibitor is durvalumab. In some embodiments, the PD-L1 inhibitor is sugemalimab. In some embodiments, the PD-L1 inhibitor is envafolimab. In some embodiments, the PD-L1 inhibitor is cosibelimab. In some embodiments, the PD-L1 inhibitor is AUNP12. In some embodiments, the PD-L1 inhibitor is CA-170. In some embodiments, the PD-L1 inhibitor is BMS-986189. In some embodiments, the PD-L1 inhibitor is BMS-936559. In some embodiments, the PD-L1 inhibitor is lodapolimab. In some embodiments, the PD-L1 inhibitor is adebrelimab. In some embodiments, the PD-L1 inhibitor is CBT-502. In some embodiments, the PD-L1 inhibitor is BGB-A33. In some embodiments, the immune checkpoint inhibitor is administered on day 1 of a 21-day cycle. In some embodiments, the immune checkpoint inhibitor is administered every 2 weeks. In some embodiments, the immune checkpoint inhibitor is administered every 4 weeks. In some embodiments, the immune checkpoint inhibitor is administered every 6 weeks.

In some embodiments, the methods described herein for the treatment of TNBC, mUC, or a Trop-2 expressing tumor do not include the further administration of an immune checkpoint inhibitor.

### Improved Patient Outcomes

By administering trilaciclib in combination with sacituzumab govitecan as described herein, one or more mechanisms leading to resistance and disease progression can be overcome, resulting in increased antigen presentation (major histocompatibility complex (MHC) class I), enhanced T cell clonality and tumor infiltration, inhibition of regulatory T cell proliferation, decreased expression of T cell exhaustion markers such as, but not limited to PD-1, cytotoxic T-lymphocyte associated protein 4 (CTLA-4), or T-cell immunoglobulin and mucin domain 3 (TIM3), stabilized expression of PD-L1 on tumor cells, promotion of dendritic cell migration, or increased T-effector cell function through high interferon-gamma (IFN-γ) production, collectively, combining to generate a robust anti-tumor T cell response. Additionally, the administration of trilaciclib in combination with sacituzumab govitecan, as described herein, can result in a reduction of the side effects attributable to sacituzumab govitecan and allow for fewer treatment interruptions. By administering trilaciclib to these difficult to treat patient subgroups, interruption in treatment and the immunosuppressive tumor microenvironment in the patient's tumor—which renders the previously administered chemotherapy and/or ICI ineffective or less effective and allows the tumor to progress—can be significantly overcome, improving the ability of the patient's immune system to reduce or control tumor burden, improving quality of life, and improving overall survival in these difficult to treat subsets of patients.

In some embodiments, the administration of a trilaciclib/sacituzumab govitecan treatment regimen described herein to the patient subgroups described herein provides enhanced anti-tumor efficacy in TNBC, mUC, or other Trop-2 overexpressing cancer patients. In some embodiments, the administration of a trilaciclib/sacituzumab govitecan treatment regimen described herein in the particular patient subgroups described above provides improved progression free survival (PFS) and/or overall survival (OS) in patients compared to those receiving sacituzumab govitecan without trilaciclib. In some embodiments, an improvement in PFS is based on per Response Evaluation Criteria in Solid Tumors 1.1 (RECIST 1.1). In some embodiments, the administration of trilaciclib in combination with sacituzumab govitecan improves the overall response rate (ORR, defined as the percentage of patients with best overall response (BOR) of complete response (CR) or partial response (PR) per RECIST v1.1) in the recipient patient population compared to a patient population receiving sacituzumab govitecan alone. In some embodiments, the administration of trilaciclib in combination with sacituzumab govitecan improves the clinical benefit rate (CBR), defined as the percentage of patients with a BOR of CR, PR, or stable disease (SD) lasting 24 weeks or longer since the first date of trilaciclib/sacituzumab govitecan administration per RECIST v1.1, in the recipient patient population compared to a patient population receiving sacituzumab govitecan alone. In some embodiments, the administration of trilaciclib in combination with sacituzumab govitecan improves the duration of response (DOR), defined as the time between first objective response of CR or PR (confirmed), as appropriate to each treatment period as described for ORR, and the first date that progressive disease is documented or death, whichever comes first, in the recipient population compared to a patient population receiving sacituzumab govitecan alone.

In some embodiments, the administration of a trilaciclib/sacituzumab govitecan treatment regimen described herein to the patient subgroups described above provides improved myelopreservation of hematopoietic stem and progenitor cells (HSPCs) and immune effector cells such as lymphocytes including T-lymphocytes. In some embodiments, the administration of a trilaciclib/sacituzumab govitecan treatment regimen described herein to the patient subgroups described above provides reduced chemotherapy-induced myelosuppression (CIM). In some embodiments, the administration of a trilaciclib/sacituzumab govitecan treatment regimen described herein provides myelopreservation of the neutrophil lineage in patients compared to those receiving sacituzumab govitecan without trilaciclib. In some embodiments, the administration of a trilaciclib/sacituzumab govitecan treatment regimen described herein provides a reduction in the duration of severe (Grade 4) neutropenia in patients compared to those receiving sacituzumab govitecan without trilaciclib. In some embodiments, the administration of a trilaciclib/sacituzumab govitecan treatment regimen described herein provides a reduction in diarrhea in patients compared to those receiving sacituzumab govitecan without trilaciclib.

In some embodiments, the administration of a trilaciclib/sacituzumab govitecan treatment regimen described herein to the patient subgroups described above provides a reduction or improvement in one or more of the following: diarrhea, the occurrence of severe neutropenia (SN); the occurrence of febrile neutropenia; the occurrence of G-CSF administration; the occurrence of Grade 3/4 decrease of hemoglobin; red blood cell (RBC) transfusions on/after Week 5; the occurrence of erythropoiesis stimulating agent (ESA) administration; the occurrence of Grade 3/4 decrease of platelets; platelet transfusions (occurrence and number of transfusions); alopecia, the occurrence of serious infections; use of IV antibiotics, in the recipient patient population compared to a patient population receiving sacituzumab govitecan without trilaciclib.

In some embodiments, the administration of a trilaciclib/sacituzumab govitecan treatment regimen described herein to the patient subgroups described above provides a reduction or improvement in one or more of the following: the occurrence and severity of adverse events (AEs) by National Cancer Institute (NCI) Common Terminology Criteria for Adverse Events (CTCAE) v5.0; the occurrence of Grade 3 or 4 abnormalities in serum chemistry laboratory parameters; and sacituzumab govitecan infusion interruptions, in the recipient patient population compared to a patient population receiving sacituzumab govitecan without trilaciclib.

In some embodiments, the administration of a trilaciclib/sacituzumab govitecan treatment regimen described herein provides a reduction in all-cause dose reductions or cycle delays and relative dose intensity for antibody drug conjugate chemotherapy. In some embodiments, the administration of a trilaciclib/sacituzumab govitecan treatment regimen described herein provides a reduction in i) hospitalizations, including but not limited to those due to all causes, febrile neutropenia/neutropenia, anemia/RBC transfusion, thrombocytopenia/bleeding and infections) or ii) antibiotic use, including but not limited to intravenous (IV), oral, and oral and IV administered antibiotics.

In some embodiments, the administration of a trilaciclib/sacituzumab govitecan treatment regimen described herein provides a reduction of chemotherapy-induced fatigue (CIF) in patients compared to those receiving antibody drug conjugate chemotherapy without trilaciclib. In some embodiments, the reduction of CIF is a reduction in the time to first confirmed deterioration of fatigue (TTCD-fatigue), as measured by the Functional Assessment of Cancer Therapy-Fatigue (FACIT-F).

In some embodiments, the administration of a trilaciclib/sacituzumab govitecan treatment regimen described herein provides an improvement to one or more of: Functional Assessment of Cancer Therapy-General (FACT-G) domain scores (physical, social/family, emotional, and functional well-being); Functional Assessment of Cancer Therapy-Anemia (FACT-An); 5-level EQ-5D (EQ-5D-5L); Patient Global Impression of Change (PGIC) fatigue item; or Patient Global Impression of Severity (PGIS) fatigue item.

### BRIEF DESCRIPTION OF THE FIGURES

FIG. 1 is a schematic of a human clinical trial assessing the safety and efficacy of trilaciclib administered with sacituzumab govitecan-hziy (Trodelvy®) in patients with locally advanced or metastatic TNBC who received at least 2 prior treatments, at least 1 in the metastatic setting. The study will include 3 study phases: Screening Phase, treatment Phase, and Survival Follow-up Phase. The treatment phase consists of 21-day cycles: Trilaciclib will be administered IV prior to sacituzumab govitecan-hziy infusions. Trilaciclib will be administered via IV prior to sacituzumab govitecan-hziy administration at 240 mg/m². Sacituzumab govitecan-hziy will be administered via IV at 10 mg/kg. The study will include 3 study phases: Screening Phase, Treatment Phase, and Survival Follow up Phase. The Treatment Phase begins on the day of the first dose of study treatment and completes at the Safety Follow-up Visit. The first Survival Follow-up assessment should occur approximately 3 months after the End of Treatment Visit. DC=discontinuation, PD=Progressive disease, PI=Principal Investigator, WD=Withdraw.

FIG. 2A-2H are a collection of tables describing the safety data of trilaciclib administered with sacituzumab govitecan-hziy (Trodelvy®) in female patients with locally advanced or metastatic TNBC who received at least 2 prior treatments, at least 1 in the metastatic setting. Unless otherwise specified, percentages are based on the number of enrolled patients who had at least one dose of any study drug (i.e., N).

FIG. 2A is a table showing female participant characteristics.

FIG. 2B is a table showing the duration of exposure and number of treatment cycles received by the study participants.

FIG. 2C is a table showing a summary of prior systemic anti-cancer therapies administered to the study participants. Medications are coded using WHO DD version Mar2021. A patient with multiple anticancer therapy entries in the same ATC (PT) is only counted once within a particular ATC (PT). Therapeutic agents are sorted by ATC followed by PT in decreasing order of frequency. ATC=anatomical therapeutic classification; PT=preferred term; WHO DD=world health organization drug dictionary.

FIG. 2D is a table showing a summary of adverse events (AEs) in the study population and the percentage of AEs that were related with trilaciclib or sacituzumab govitecan-hziv administration. Related refers to those events that are Possibly, Probably, or Definitely Related, as assessed by investigators. AEs are coded using MedDRA Version 24.0. AEs with missing information concerning relationship, severity, or action taken are excluded from any specific analysis related to those parameters, however, these are included where appropriate in the overall summary. Adverse Events=AE; MedDRA=medical dictionary for regulatory activities.

FIG. 2E is a table summarizing the occurrence of myelosuppression endpoints related to the 3 blood lineages (white blood cells, red blood cells, and platelets) in the study population. G-CSF=granulocyte colony stimulating factor; ESA=erythropoietin stimulating factor.

FIG. 2F is a table summarizing AEs and AEs related to study drugs trilaciclib or sacituzumab govitecan-hziv by system organ class and preferred term in the study population. AEs are defined as those AEs that started on or after the day of the first dose of study drug through the Safety Follow-up Visit. AEs with an unknown/not reported onset date are also included. AEs are coded using MedDRA version 24.0. A patient with multiple TEAE entries in the same PT is only counted once within a particular preferred term at the highest CTCAE grade. Unless otherwise specified, percentages are based on the number of enrolled patients who had at least one dose of any study drug (i.e. N). Adverse Events=AE; MedDRA=medical dictionary for regulatory activities; CTCAE=common terminology criteria for adverse events.

FIG. 2G is a table summarizing alopecia, neutropenia, and neutrophil count decreased AEs in the study population. Adverse Events=AE.

FIG. 2H is a table summarizing best overall response based on derived assessments (confirmed) in the study population. [a] The 95% CI for the proportion is calculated using the exact Clopper-Pearson method. [b] SD lasting 24 weeks or longer since the first date of study drug administration per RECIST v1.1. Overall response is derived based on Response Evaluation Criteria in Solid Tumors (RECIST), Version 1.1. CI=confidence interval.

### DETAILED DESCRIPTION OF THE INVENTION

### Definitions

Unless otherwise defined, all technical and scientific terms used herein have the same meaning as commonly understood by one of ordinary skill in the art to which this application belongs. In the specification, singular forms also include the plural unless the context clearly dictates otherwise. Although methods and materials similar or equivalent to those described herein can be used in the practice and testing of the present application, suitable methods and materials are described below. All publications, patent applications, patents, and other references mentioned herein are incorporated by reference. The references cited herein are not admitted to be prior art to the claimed application. In the case of conflict, the present specification, including definitions, will control. In addition, the materials, methods, and examples are illustrative only and are not intended to be limiting.

Compounds are described using standard nomenclature. Unless defined otherwise, all technical and scientific terms used herein have the same meaning as is commonly understood by one of skill in the art to which this invention belongs.

In some embodiments of each compound described herein, the compound may be in the form of a racemate, enantiomer, mixture of enantiomers, diastereomer, mixture of diastereomers, tautomer, N-oxide, or isomer, such as a rotamer, as if each is specifically described unless specifically excluded by context.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individual recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and do not pose a limitation on the scope of the invention unless otherwise claimed.

In some embodiments of each compound described herein, the compound may be in the form of a tautomer, N-oxide, or isomer, such as a rotamer, as if each is specifically described unless specifically excluded by context.

An “effective amount” as used herein, means an amount which provides a therapeutic or prophylactic benefit.

The term “about” as used herein, means plus or minus 10%.

To “treat” a disease as the term is used herein, means to reduce the frequency or severity of at least one sign or symptom of a disease, disorder, or side-effect experienced by a patient (i.e., palliative treatment) or to decrease a cause or effect of the disease, disorder (i.e., disease-modifying treatment), or side effect experienced by a patient as a result of the administration of a therapeutic agent.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, “pharmaceutical compositions” are compositions comprising at least one active agent, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985). Wherein the methods described herein identify the administration of a particular compound, it is understood that administration of the compound's pharmaceutically acceptable salt, if applicable, is encompassed as an embodiment.

As used herein, the term “prodrug” means a compound which when administered to a host in vivo is converted into the parent drug. As used herein, the term “parent drug” means any of the presently described chemical compounds that are useful to treat any of the disorders described herein, or to control or improve the underlying cause or symptoms associated with any physiological or pathological disorder described herein in a host, typically a human. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Nonlimiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

The term “carrier” applied to pharmaceutical compositions/combinations of the invention refers to a diluent, excipient, or vehicle with which an active compound is provided.

A “pharmaceutically acceptable excipient” means an excipient that is useful in preparing a pharmaceutical composition/combination that is generally safe, and neither biologically nor otherwise inappropriate for administration to a host, typically a human.

In non-limiting embodiments, trilaciclib can be used in a form that has at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into trilaciclib for use in the invention include isotopes of hydrogen, carbon, nitrogen, oxygen, phosphorous, fluorine, chlorine and iodine such as 2H, 3H, 11C, 13C, 14C, 15N, 18F 31P, 32P, 35S, 36CI, and 125I respectively. In one non-limiting embodiment, isotopically labelled compounds can be used in metabolic studies (with 14C), reaction kinetic studies (with, for example 2H or 3H), detection or imaging techniques, such as positron emission tomography (PET) or single-photon emission computed tomography (SPECT) including drug or substrate tissue distribution assays, or in radioactive treatment of patients. In particular, an 18F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively, or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

The “patient,” “host,” or “subject” treated is typically a human patient, although it is to be understood the methods described herein are effective with respect to other animals, such as mammals. More particularly, the term patient can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like. In particular embodiments, the patient, host, or subject is a human patient.

As generally contemplated herein, the term “hematopoietic stem and progenitor cells” (HSPCs) includes, but are not limited to, long term hematopoietic stem cells (LT-HSCs), short term hematopoietic stem cells (ST-HSCs), hematopoietic progenitor cells (HPCs), multipotent progenitors (MPPs), oligodendrocyte pre-progenitors (OPPs), monocyte progenitors, granulocyte progenitors, common myeloid progenitors (CMPs), common lymphoid progenitors (CLPs), granulocyte-monocyte progenitors (G1VIPs), granulocyte progenitors, monocyte progenitors, and megakaryocyte-erythroid progenitors (MEPs), megakaryocyte progenitors, erythroid progenitors, HSC/MPP s (CD45dim/CD34+/CD38−), OPPs (CD45dim/CD34+/CD38+), monocyte progenitors

(CD45+/CD14+/CD11b+), granulocyte progenitors (CD45+/CD14−/CD11b+), erythroid progenitors (CD45−/CD71+), and megakaryocyte progenitors (CD45+/CD61+).

The term “immune effector cell” generally refers to an immune cell that performs one or more specific functions. Immune effector cells are known in the art and include for example, but are not limited to, T-cells, including Naïve T-cells, Memory T-cells, Activated T-cells (T helper (CD4+) and Cytotoxic T cells (CD8+)), TH1 activated T-cells, TH2 activated T-cells, TH17 activated T-cells, Naïve B cells, Memory B cells, plasmablasts, dendritic cells, monocytes, and natural killer (NK) cells.

As used herein, the term “immune checkpoint inhibitor (ICI)” refers to therapy targeting immune checkpoint proteins, key regulators of the immune system that when expressed can dampen the immune response to an immunologic stimulus. Some cancers express ligands for the checkpoint inhibitors and can protect themselves from attack by binding to immune checkpoint targets. ICIs block inhibitory checkpoints, restoring immune system function. ICIs include those targeting immune checkpoint proteins such as PD-1, PD-1 Ligand-1 (PD-L1), PD-1 Ligand-2 (PD-L2), CTLA-4, LAG-3, TIM-3, and V-domain Ig suppressor of T-cell activation (VISTA), B7-H3/CD276, indoleamine 2,3-dioxygenase (IDO), killer immunoglobulin-like receptors (KIRs), carcinoembryonic antigen cell adhesion molecules (CEACAM) such as CEACAM-1, CEACAM-3, and CEACAM-5, sialic acid-binding immunoglobulin-like lectin 15 (Siglec-15), T cell immunoreceptor with Ig and ITIM domains (TIGIT), and B and T lymphocyte attenuator (BTLA) protein. Immune checkpoint inhibitors are known in the art.

### Trop-2 Expressing Cancers

Trophoblast cell surface antigen 2 (Trop-2) is a glycoprotein that spans the epithelial membrane surface and plays a role in cell self-renewal, proliferation, and transformation (Zaman et al., Targeting Trop-2 in solid tumors: future prospects. Onco Targets Ther. 2019;12:1781-1790).

Under physiological conditions, Trop-2 plays an essential role in embryonic development, placental tissue formation, embryo implantation, stem cell proliferation, and organ development (Shvartsur et al., Trop2 and its overexpression in cancers: regulation and clinical/therapeutic implications. Genes Cancer. 2015;6(3-4):84-105). A low basal expression level of Trop-2 is found on the surface of multiple normal epithelial tissues, including skin and oral mucosa (Strop P, Tran

TT, Dorywalska M, et al. RN927C, a site-specific Trop-2 antibody-drug conjugate (ADC) with enhanced stability, is highly efficacious in preclinical solid tumor models. Mol Cancer Ther. 2016;15(11):2698-2708). Trop-2 can promote tumor growth and its overexpression is common in many types of malignant epithelial tumors (Goldenberg D M, Stein R, Sharkey R M. The emergence of trophoblast cell-surface antigen 2 (TROP-2) as a novel cancer target. Oncotarget. 2018;9(48):28989-29006). Overexpression of Trop-2 accelerates the cancer cell cycle and drives cancer growth. Trop2 overexpression is associated with decreased patient survival as well as increased tumor aggressiveness and metastasis in many cancers. In some embodiments, patients with advanced/metastatic cancers that overexpress Trop-2 are administered trilaciclib, or a pharmaceutically acceptable salt thereof, in combination with the antibody drug conjugate sacituzumab govitecan in a specifically timed administrative protocol described herein. In some embodiments, the advanced/metastatic cancers that overexpress Trop-2 are selected from the group consisting of breast cancer, cervical cancer, colon or colorectal cancer, endometroid endometrial cancer, esophageal cancer, gastric cancer, gliomas, hilar cholangiocarcinoma, squamous cell carcinoma of the oral cavity, gastrointestinal cancer, chronic lymphocytic lymphoma, extranodal NK/T-cell lymphoma, non-Hodgkin's lymphoma, Raji Burkitt lymphoma, small-sized pulmonary adenocarcinoma, ovarian cancer, pancreatic cancer, prostate cancer, stomach carcinoma, thyroid carcinoma, urinary bladder carcinoma, uterine cancer, and lung cancer, including small cell lung cancer and non-small cell lung cancer.

In some embodiments, the Trop-2 expressing tumor to be treated is non-small cell lung cancer (NSCLC). NSCLC makes up nearly 85% of all lung cancers diagnosed. Common NSCLC types include adenocarcinoma, which generally presents with glandular differentiation, squamous cell carcinoma, which generally presents with squamous differentiation (keratinization), and large cell carcinoma, which generally presents as large and poorly-differentiated.

### Triple Negative Breast Cancer

Triple-negative Breast Cancer (TNBC) is a highly aggressive breast cancer subtype that accounts for 15-20% of breast cancer cases annually and 25% of all breast cancer deaths. TNBC has been characterized by several aggressive clinicopathologic features, including onset at a younger age; large, high-grade tumors; and a propensity for visceral metastasis (Cheang et al., Basal-like breast cancer defined by five biomarkers has superior prognostic value than triple-negative phenotype. Clin Cancer Res. 2008;14(5):1368-76.; Foulkes et al., Triple-negative breast cancer. N Engl J Med. 2010 Nov 11;363(20):1938-48).

A breast cancer is generally classified as TNBC based on local ER-negative, progesterone receptor (PR)-negative, HER2-negative status, which can be determined through a histological or cytological hormone receptor immunohistochemistry (IHC) assessment for estrogen and progesterone (defined as <1% nuclei staining), and by IHC [0 or 1+] OR in situ hybridization [ratio <2.0] OR average gene copy number of <4 signals/nucleus) for HER2-negative, non-overexpression (per 2018 American Society of Clinical Oncology and the College of American Pathologists (ASCO CAP) criteria).

### Metastatic Urothelial Cancer

Metastatic urothelial (transitional cell) carcinoma (mUC) is the predominant histologic type of bladder cancer in the United States and Europe, where it accounts for 90 percent of all bladder cancers. Bladder cancer is the 6th most commonly occurring cancer in men and the 17th most commonly occurring cancer in women globally. Bladder cancer is the most common malignancy involving the urinary system. Bladder cancer can be categorized as non-muscle invasive, muscle invasive, or metastatic. Approximately 25 percent of patients will have muscle-invasive disease and either present with or later develop metastases. Systemic chemotherapy is the standard approach for the initial treatment of patients with inoperable locally advanced or metastatic urothelial malignancies. Although initial response rates are high, the median survival with multiagent chemotherapy is approximately 15 months

### PD-L1 Status

In some aspects, the Trop-2 overexpressing cancer to be treated is PD-L1 positive. In alternative aspects, the Trop-2 overexpressing cancer to be treated is PD-L1 negative.

PD-L1 is a transmembrane protein that down-regulates immune responses through binding to its two inhibitory receptors, programmed death-1 (PD-1) and B7.1. PD-1 is an inhibitory receptor expressed on T cells following T-cell activation, which is sustained in states of chronic stimulation such as in chronic infection or cancer (Blank, C and Mackensen, A, Contribution of the PD-L1/PD-1 pathway to T-cell exhaustion: an update on implications for chronic infections and tumor evasion. Cancer Immunol Immunother, 2007. 56(5): p. 739-745). Binding of PD-Ll with PD-1 inhibits T cell proliferation, cytokine production and cytolytic activity, leading to the functional inactivation or exhaustion of T cells. B7.1 is a molecule expressed on antigen presenting cells and activated T cells. PD-L1 binding to B7.1 on T cells and antigen presenting cells can mediate down-regulation of immune responses, including inhibition of T-cell activation and cytokine production (see Butte M J, Keir M E, Phamduy T B, et al. Programmed death-1 ligand 1 interacts specifically with the B7-1 costimulatory molecule to inhibit T cell responses. Immunity. 2007;27(1):111-122). PD-L1 expression has been observed in immune cells and tumor cells. See Dong H, Zhu G, Tamada K, Chen L. B7-H1, a third member of the B7 family, co-stimulates T-cell proliferation and interleukin-10 secretion. Nat Med. 1999;5(12):1365-1369; Herbst R S, Soria J C, Kowanetz M, et al. Predictive correlates of response to the anti-PD-L1 antibody MPDL3280A in cancer patients. Nature. 2014;515(7528):563-567. Aberrant expression of PD-L1 on tumor cells has been reported to impede anti-tumor immunity, resulting in immune evasion.

PD-L1 expression can be determined by methods known in the art. For example, PD-L1 expression can be detected using PD-L1 IHC 22C3 pharmDx, the FDA-approved in vitro diagnostic immunohistochemistry (IHC) test developed by Dako and Bristol-Meyers Squibb as a companion test for treatment with pembrolizumab. This is qualitative assay using Monoclonal Mouse Anti-PD-L1, Clone 22C3 PD-L1 and EnVision FLEX visualization system on Autostainer Lin 48 to detect PD-L1 in formalin-fixed, paraffin-embedded (FFPE) human cancer tissue. Expression levels can be measured using the tumor proportion score (TPS), which measures the percentage of viable tumor cells showing partial or complete membrane staining. Staining can show PD-L1 expression from 1% to 100%.

PD-L1 expression can also be detected using PD-L1 IHC 28-8 pharmDx, the FDA-approved in vitro diagnostic immunohistochemistry (IHC) test developed by Dako and Merck as a companion test for treatment with nivolumab. This qualitative assay uses the Monoclonal rabbit anti-PD-L1, Clone 28-8 and EnVision FLEX visualization system on Autostainer Lin 48 to detect PD-L1 in formalin-fixed, paraffin-embedded (FFPE) human metastatic urothelial cancer tissue.

Other commercially available tests for PD-L1 detection include the Ventana SP263 assay (developed by Ventana in collaboration with AstraZeneca) that utilizes monoclonal rabbit anti-PD-L1, Clone SP263 and the Ventana SP142 Assay (developed by Ventana in collaboration with Genentech/Roche) that uses rabbit monoclonal anti-PD-L1 clone SP142. Determination of PD-L1 status is indication-specific, and evaluation is based on either the proportion of tumor area occupied by PD-L1 expressing tumor-infiltrating immune cells (% IC) of any intensity or the percentage of PD-L1 expressing tumor cells (% TC) of any intensity. For example, PD-L1 positive status in TNBC is considered >1% IC and in mUC is considered ≥50% TC or ≥10% IC.

In some embodiments, the TNBC has a PD-L1 positive status ≥1% IC.

In some embodiments, the mUC patient being treated in the second-line or third-line antibody drug conjugate chemotherapeutic protocol has a documented PD-L1 status positive mUC. In some embodiments, the patient being treated in the second-line or third-line antibody drug conjugate chemotherapeutic protocol has a documented PD-L1 status positive mUC of >10% PD-L1 staining tumor-infiltrating immune cells or >50% of tumor cells as confirmed by an in vitro diagnostic (IVD) assay, for example, the Ventana SP-142 assay or other suitable assay. In some embodiments, the patient being treated in the second-line or third-line antibody drug conjugate chemotherapeutic protocol has a documented PD-L1 status mUC with >20% PD-L1 staining of tumor cells as confirmed by an in vitro diagnostic (IVD) assay, for example, the Ventana SP-142 assay or other suitable assay. In an alternative embodiment, the patient being treated in the second-line or third-line antibody drug conjugate chemotherapeutic protocol has a documented PD-L1 status mUC with ≥1% PD-L1 staining of tumor cells as determined by an FDA-approved test. In an alternative embodiment, the patient being treated in the second-line or third-line antibody drug conjugate chemotherapeutic protocol has a documented PD-L1 status negative mUC.

### CDK4/6 Status

As provided herein, in some embodiments, the Trop-2 overexpressing cancer to be treated is CDK4/6-negative or CDK4/6 replication independent. In alternative embodiments, the Trop-2 overexpressing cancer to be treated is CDK4/6-positive or CDK4/6 replication dependent. In still other alternative embodiments, the Trop-2 overexpressing cancer to be treated is CDK4/6 indeterminate.

CDK4/6 replication independent cancers generally have a retinoblastoma gene (Rb 1) aberration. The gene product of Rb1—Rb-protein—is a downstream target of CDK4/6. RBI is commonly dysregulated in cancer cells through deletion, mutation or epigenetic modification resulting in loss of RB expression, as well as by aberrant CDK kinase activity leading to excessive phosphorylation and inactivation of RB function (Chen et al. Novel RB1-Loss Transcriptomic Signature Is Associated with Poor Clinical Outcomes across Cancer Types. Clin Cancer Res. 2019;25(14); Sherr, C. J., and McCormick, F. The RB and p53 pathways in cancer. Cancer Cell, 2002;2:103 12.). CCNE1/2 (cyclin E) is part of a parallel pathway that provides functional redundancy with CDK4/6 and helps to transition cells from the G1 to S phase. Overexpression will decrease the reliance on the CDK4/6 pathway leading to CDK4/6 independence (Turner et al., Cyclin E1 Expression and Palbociclib Efficacy in Previously Treated Hormone Receptor—Positive Metastatic Breast Cancer. J Clin Oncol. 2019;37(14):1169-78.). Therefore, a tumor with either CCNE1/2 amplification or RB loss will generally be considered “CDK4/6 independent”.

Cancers that are CDK4/6 replication dependent require the activity of CDK4/6 for replication or proliferation. CDK 4/6 replication dependent TNBCs generally have an intact and functional Rb pathway and/increased expression of CDK4/6 activators (cyclin D), and/or a d-type cyclin activating features (DCAF)—including CCND1 translocation, CCND1-3 3′UTR loss, and amplification of CCND2 or CCND3 (see Gong et al. Genomic aberrations that activate D-type cyclins are associated with enhanced sensitivity to the CDK4 and CDK5 inhibitor abemaciclib. Cancer Cell. 2017;32(6):761-76). Tumors that are wildtype for RB and CCNE1/2 as well as have one of the DCAF described above are generally classified as “CDK4/6 dependent”.

Tumors that cannot be classified as either CDK4/6-replication dependent or CDK4/6-replication independent are generally classified as “CDK4/6 indeterminate” since they cannot be confirmed as CDK4/6 dependent or independent.

In some embodiments, the Trop-2 overexpressing cancer is classified as CDK4/6-replication dependent. In some embodiments, the Trop-2 overexpressing cancer is classified as CDK4/6-replication independent. In some embodiments, the Trop-2 overexpressing cancer is classified as CDK4/6 indeterminate.

Methods of determining CDK4/6 genetic signature analysis are known in the art and involve the utilization of tumor tissue collected from a patients' biopsy (e.g., TNBC or mUC primary or metastatic site) and are described in Shapiro GI. Genomic biomarkers predicting response to selective CDK4/6 inhibition: Progress in an elusive search. Cancer Cell. 2017; 32(6):721-3 and Gong et al. Genomic aberrations that activate D-type cyclins are associated with enhanced sensitivity to the CDK4 and CDK5 inhibitor abemaciclib. Cancer Cell. 2017; 32(6):761-76.

In some embodiments, the patient receiving trilaciclib in combination with sacituzumab govitecan, has a CDK4/6 independent TNBC having at least one of the following:
a. a CCNE1 amplification;
b. a CCNE2 amplification; or
c. a Retinoblastoma protein 1 (Rb 1) loss, which is defined as i) homozygous deletion, ii) a frameshift mutation, or iii) a stop-gained mutation (i.e., a mutation that results in a premature termination codon (a stop codon was gained)).

In some embodiments, the patient receiving trilaciclib in combination with sacituzumab govitecan, has a CDK4/6 dependent TNBC which does not have
1) at least one of the following:
a. a CCNE1 amplification;
b. a CCNE2 amplification; or
c. a Retinoblastoma protein 1 (Rb 1) loss, which is defined as i) homozygous deletion, ii) a frameshift mutation, or iii) a stop-gained mutation (i.e., a mutation that results in a premature termination codon (a stop codon was gained));
2) does have a wild type: i) CCNE1; ii) CCNE2; and iii) RB1; and
3) has at least one of the following D-cyclin activating features: i) CCND2 amplification; ii) CCND3 amplification; and iii) CCD1-3 3′ UTR loss defined as a homozygous or heterozygous deletion of any of these UTRs.

In some embodiments, the patient receiving trilaciclib in combination with sacituzumab govitecan, has a CDK4/6 independent mUC having at least one of the following:
d. a CCNE1 amplification;
e. a CCNE2 amplification; or
f. a Retinoblastoma protein 1 (Rb 1) loss, which is defined as i) homozygous deletion, ii) a frameshift mutation, or iii) a stop-gained mutation (i.e., a mutation that results in a premature termination codon (a stop codon was gained)).

In some embodiments, the patient receiving trilaciclib in combination with sacituzumab govitecan-hziy, has a CDK4/6 dependent mUC which does not have
1) at least one of the following:
a. a CCNE1 amplification;
b. a CCNE2 amplification; or
c. a Retinoblastoma protein 1 (Rb 1) loss, which is defined as i) homozygous deletion, ii) a frameshift mutation, or iii) a stop-gained mutation (i.e., a mutation that results in a premature termination codon (a stop codon was gained));
2) does have a wild type: i) CCNE1; ii) CCNE2; and iii) RBI; and
3) has at least one of the following D-cyclin activating features: i) CCND2 amplification; ii) CCND3 amplification; and iii) CCD1-3 3′ UTR loss defined as a homozygous or heterozygous deletion of any of these UTRs.

### Improved Antibody Drug Conjugate Chemotherapeutic Protocols

### Trilaciclib

Trilaciclib (2′-((5-(4-methylpiperazin-1-yl)pyridin-2-yl)amino)-7′,8′-dihydro-6′H-spiro(cyclohexane-1,9′-pyrazino(1′,2′:1,5)pyrrolo(2,3-d)pyrimidin)-6′-one) is a highly selective CDK4/6 inhibitor having the structure:

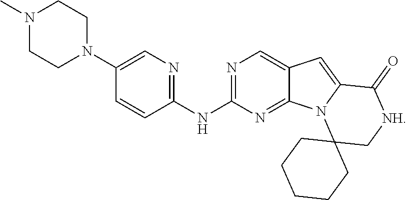 [SMILES: CN1CCN(c2ccc(Nc3ncc4cc5n(c4n3)C3(CCCCC3)CNC5=O)nc2)CC1]

As provided herein, trilaciclib or its pharmaceutically acceptable salt, composition, isotopic analog, or prodrug thereof is administered in a suitable carrier. Trilaciclib is described in US 2013-0237544, incorporated herein by reference in its entirety. Trilaciclib can be synthesized as described in US 2019-0135820, incorporated herein by reference in its entirety. Trilaciclib can be administered in any manner that achieves the desired outcome, including systemically, parenterally, intravenously, intramuscularly, subcutaneously, or intradermally. For injection, trilaciclib may be provided, in some embodiments, for example, as a 300 mg/vial as a sterile, lyophilized, yellow cake providing 300 mg of trilaciclib (equivalent to 349 mg of trilaciclib dihydrochloride, dihydrate). The product, for example, may be supplied in single-use 20-mL clear glass vials which does not contain a preservative. For example, prior to administration, trilaciclib for injection, 300 mg/vial may be reconstituted with 19.5 ml of 0.9% sodium chloride injection or 5% dextrose injection. This reconstituted solution has a trilaciclib concentration of 15 mg/mL and would typically be subsequently diluted prior to intravenous administration. Trilaciclib can be administered intravenously as described herein.

Trilaciclib for use in the present invention may be in the form of a salt, for example a dihydrochloride salt. In certain aspects of the invention Trilaciclib is a crystalline dihydrochloride salt which can be reconstituted for intravenous delivery. In certain embodiments Trilaciclib is a crystalline dihydrochloride salt dihydrate which can be reconstituted for intravenous delivery.

In certain embodiments, trilaciclib is in the form a solvate with solvents (including water). The term “solvate” refers to a molecular complex of trilaciclib (including a salt thereof) with one or more solvent molecules. Non-limiting examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g., D20, d6-acetone, d6-DMSO. A solvate can be in a liquid or solid form.

In certain embodiments, trilaciclib is in the form of a dihydrochloride optionally as a hydrate. For example, trilaciclib can be used in the present invention as a dihydrochloride, dihydrate or as a pharmaceutical composition formed from trilaciclib dihydrochloride, dihydrate.

In some embodiments, trilaciclib is administered at between about 180 mg/m² and 300 mg/m². In some embodiments, trilaciclib is administered at about 180, 185, 190, 195, 200, 205, 210, 215, 220, 225, 230, 235, 240, 245, 250, 255, 260, 265, 270, 275, or about 280 mg/m². In some embodiments, trilaciclib is administered at least 180, 185, 190, 195, 200, 205, 210, 215, 220, 225, 230, 235, or 240 mg/m². In some embodiments, trilaciclib is administered at about 240 mg/m², prior to administration of for example, sacituzumab govitecan, for example prior to about 4 hours or less, for example about 4 hours or less, 3 hours or less, 2 hours or less, about 1 hour or less, or about 30 minutes prior to administration of sacituzumab govitecan. In some embodiments, trilaciclib is administered intravenously over a period of about 30 minutes. In some embodiments, trilaciclib is completely administered prior to administration of sacituzumab govitecan.

As provided herein, for the treatment of advanced/metastatic TNBC, trilaciclib is administered on day 1 and day 8 of each 21-day cycle, or as otherwise provided herein. Trilaciclib is administered prior to the initiation of administration of sacituzumab govitecan, generally via intravenous injection/infusion over about 30 minutes about 4 hours or less, for example about 3 hours or less, 2 hours or less, 1 hour or less, or about 30 minutes prior to the initiation of the administration of sacituzumab govitecan in the protocol. In some embodiments, the trilaciclib is completely administered on day 1 and day 8 no more than 4 hours prior to initiation of the administration of sacituzumab govitecan.

In alternative embodiments, for the treatment of advanced/metastatic TNBC, trilaciclib is administered on day 1, day 8, and day 15 of each 21-day cycle, or as otherwise provided herein. Trilaciclib is administered prior to the initiation of administration of sacituzumab govitecan on days 1 and day 8, generally via intravenous injection/infusion over about 30 minutes about 4 hours or less, for example about 3 hours or less, 2 hours or less, 1 hour or less, or about 30 minutes prior to the initiation of the administration of sacituzumab govitecan in the protocol. In some embodiments, the trilaciclib is completely administered on day 1 and day 8 no more than 4 hours prior to initiation of the administration of sacituzumab govitecan.

In an alternative embodiment, as provided herein, for the treatment of advanced/metastatic urothelial cancer, trilaciclib is administered on day 1 and day 8 of each 21-day cycle, or as otherwise provided herein. Trilaciclib is administered prior to the initiation of administration of sacituzumab govitecan, generally via intravenous injection/infusion over about 30 minutes about 4 hours or less, for example about 3 hours or less, 2 hours or less, 1 hour or less, or about 30 minutes prior to the initiation of the administration of sacituzumab govitecan in the protocol. In some embodiments, the trilaciclib is completely administered on day 1 and day 8 no more than 4 hours prior to initiation of the administration of sacituzumab govitecan.

In alternative embodiments, as provided herein, for the treatment of advanced/metastatic urothelial cancer, trilaciclib is administered on day 1, day 8, and day 15 of each 21-day cycle, or as otherwise provided herein. Trilaciclib is administered prior to the initiation of administration of sacituzumab govitecan on day 1 and day 8, generally via intravenous injection/infusion over about 30 minutes about 4 hours or less, for example about 3 hours or less, 2 hours or less, 1 hour or less, or about 30 minutes prior to the initiation of the administration of sacituzumab govitecan in the protocol. In some embodiments, the trilaciclib is completely administered on day 1 and day 8 no more than 4 hours prior to initiation of the administration of sacituzumab govitecan.

In an alternative embodiment, as provided herein, for the treatment of a Trop-2 overexpressing cancer, trilaciclib is administered on day 1 and day 8 of each 21-day cycle, or as otherwise provided herein. Trilaciclib is administered prior to the initiation of administration of sacituzumab govitecan, generally via intravenous injection/infusion over about 30 minutes about 4 hours or less, for example about 3 hours or less, 2 hours or less, 1 hour or less, or about 30 minutes prior to the initiation of the administration of sacituzumab govitecan in the protocol. In some embodiments, the trilaciclib is completely administered on day 1 and day 8 no more than 4 hours prior to initiation of the administration of sacituzumab govitecan. In some embodiments, the Trop-2 overexpressing cancer is non-small cell lung cancer (NSCLC). In a particular embodiment, the NSCLC is metastatic or advanced NSCLC. In some embodiments, the NSCLC has progressed on or after platinum-based chemotherapy and PD-1 or PD-L1 inhibitor therapy received either in combination or sequentially.

In alternative embodiments, as provided herein, for the treatment of a Trop-2 overexpressing cancer, trilaciclib is administered on day 1, day 8, and day 15 of each 21-day cycle, or as otherwise provided herein. Trilaciclib is administered prior to the initiation of administration of sacituzumab govitecan on day 1 and day 8, generally via intravenous injection/infusion over about 30 minutes about 4 hours or less, for example about 3 hours or less, 2 hours or less, 1 hour or less, or about 30 minutes prior to the initiation of the administration of sacituzumab govitecan in the protocol. In some embodiments, the trilaciclib is completely administered on day 1 and day 8 no more than 4 hours prior to initiation of the administration of sacituzumab govitecan. In some embodiments, the Trop-2 overexpressing cancer is non-small cell lung cancer (NSCLC). In a particular embodiment, the NSCLC is metastatic or advanced NSCLC. In some embodiments, the NSCLC has progressed on or after platinum-based chemotherapy and PD-1 or PD-L1 inhibitor therapy received either in combination or sequentially.

In an alternative embodiment, a different CDK4/6 inhibitor is administered. For example, in alternative embodiments, the CDK4/6 inhibitor used instead of trilaciclib in the protocols described herein is ribociclib (Novartis), palbociclib (Pfizer), or abemaciclib (Eli Lily), or a pharmaceutically acceptable salt thereof. In an additional alternative embodiment, the CDK4/6 inhibitor is lerociclib, which has the structure:

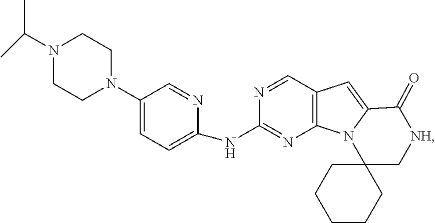 [SMILES: CC(C)N1CCN(c2ccc(Nc3ncc4cc5n(c4n3)C3(CCCCC3)CNC5=O)nc2)CC1]

or a pharmaceutically acceptable composition, salt, isotopic analog, or prodrug thereof, which is described in US 2013-0237544, incorporated herein by reference in its entirety, and can be synthesized as described in US 2019-0135820, incorporated herein by reference in its entirety. In some embodiments, lerociclib is administered as a pharmaceutically acceptable salt, for example, the dihydrocloride salt.

In an additional alternative embodiment, the CDK4/6 inhibitor has the structure:

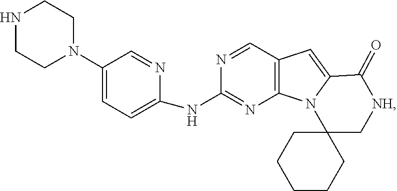 [SMILES: O=C1NCC2(CCCCC2)n2c1cc1cnc(Nc3ccc(N4CCNCC4)cn3)nc12]

or a pharmaceutically acceptable composition, salt, isotopic analog, or prodrug thereof, which is described in US 2013-0237544, incorporated herein by reference in its entirety, and can be synthesized as described in US 2019-0135820, incorporated herein by reference in its entirety.

### Sacituzumab Govitecan

Sacituzumab govitecan (Trodelvy ®) is an antibody drug conjugate (ADC) composed of the humanized RS7 (hRS7) anti-Trop-2 monoclonal antibody and a cleavable CL2A linker coupled to a cytotoxic payload SN-38, an active metabolite of the topoisomerase I inhibitor irinotecan. The FDA has designated Trodelvy sacituzumab govitecan-hziy to distinguish it from biosimilar molecules. The methods described herein shall be understood to include sacituzumab govitecan-hziy and all biosimilars thereof and the use of term sacituzumab govitecan-hziy is intended to be non-limiting with respect to any biosimilars thereof. Sacituzumab govitecan-hziy has a molecular weight of approximately 160 kilodaltons. Sacituzumab govitecan-hziy has the following chemical structure:

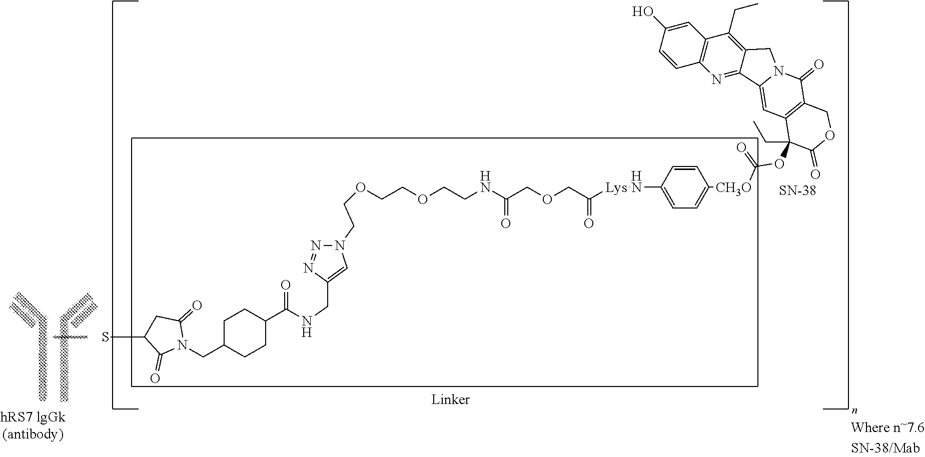

### Where n-7.6 SN-38/Mab.

Trop-2 is a calcium signal transducer overexpressed in many epithelial cancers, including breast and urothelial cancer, and implicated in the promotion of cellular proliferation, survival, and invasion. High levels of Trop-2 expression are associated with worse survival in these indications. Sacituzumab govitecan-hziy has a high site-specific coupling of 7.6 molecules of SN-38 per monoclonal antibody without altering pharmacokinetics or reducing therapeutic index of the conjugated antibody. This allows for the delivery of high, localized concentrations of SN-38 to tumor tissue. Following binding to Trop-2, the ADC is internalized and trafficked intracellularly to lysosomes. SN-38 is released throughout antibody degradation followed by hydrolysis of the linker at low pH that can be found within the lysosomes as well as extracellularly in the tumor microenvironment.

In 2020, accelerated approval was granted by FDA for sacituzumab govitecan-hziy (Trodelvy®) for the treatment of adult patients with metastatic TNBC who have received at least two prior therapies for metastatic disease. The accelerated approval was based on results from the Phase 2 IMMU-132-01 trial where patients (N=108) treated with sacituzumab govitecan-hziy had an ORR of 33.3%, median duration of response (DOR) of 7.7 months (95% CI=4.9-10.8 months), and 55.5% and 16.7% of patients having a DOR of ≥6 months and ≥12 months, respectively (Bardia, 2019). In 2021, full approval was granted by FDA based on the results from the Phase 3 ASCENT trial (Bardia, 2021). Median PFS for patients receiving sacituzumab govitecan-hziy was 4.8 months (95% CI=4.1-5.8 months) compared with 1.7 months (95% CI=1.5-2.5 months) in those receiving physician's choice of single-agent chemotherapy (HR=0.43, 95% CI 0.35-0.54, P<0.0001) and median overall survival (OS) was 11.8 months (95% CI=10.5-13.8 months) and 6.9 months (95% CI=5.9-7.6 months), respectively (HR=0.51, 95% CI=0.41-0.62, P<0.0001) (Trodelvy Package Insert, 2021).

In April 2021, accelerated approval was granted by FDA for sacituzumab govitecan-hziy (Trodelvy®) for the treatment of patients with locally advanced or metastatic urothelial cancer (mUC) who previously received a platinum-containing chemotherapy and either a PD-1 or a PD-L1 inhibitor. Efficacy and safety were evaluated in TROPHY (IMMU-132-06; NCT03547973), a single-arm, multicenter trial that enrolled 112 patients with locally advanced or mUC who received prior treatment with a platinum-containing chemotherapy and either a PD-1 or PD-L1 inhibitor. Patients received sacituzumab govitecan, 10 mg/kg intravenously, on days 1 and 8 of a 21-day treatment cycle. The main efficacy endpoints were objective response rate (ORR) and duration of response (DOR), evaluated by independent review using RECIST 1.1 criteria. The confirmed ORR was 27.7% (95% CI:19.6, 36.9) with 5.4% complete responses and 22.3% partial responses. The median DOR was 7.2 months (n=31; 95% CI: 4.7, 8.6; range 1.4+, 13.7).

Per Warnings and Precautions in the prescribing information for sacituzumab govitecan-hziy (Trodelvy® Package Insert, 2021), the following are important risks related to sacituzumab govitecan-hziy use:
Severe or life-threatening neutropenia (boxed warning);
Severe diarrhea (boxed warning);
Hypersensitivity and infusion-related reactions, including severe anaphylactic reactions;
Nausea and vomiting;
Patients with Reduced UGT1A1 Activity: Individuals who are homozygous for the uridine diphosphate-glucuronosyl transferase 1A1 (UGT1A1)*28 allele are at increased risk for neutropenia, febrile neutropenia, and anemia; and,
Embryo-fetal toxicity.

Although the development of chemotherapy-induced myelosuppression (CIM) is potentially problematic in all chemotherapy, it is particularly problematic during sacituzumab govitecan treatment, as the treatment is generally administered after significant damage to the hematological cell population from previous courses of treatment has occurred. Patients who develop CIM are more likely to experience infections, sepsis, bleeding, and fatigue, often leading to the need for hospitalizations, hematopoietic growth factor support, transfusions (red blood cells [RBCs] and/or platelets), and even death (see, e.g., Gustinetti et al., Bloodstream infections in neutropenic cancer patients: A practical update. Virulence. 2016; 7(3): 280-97; Li et al., Relationship between severity and duration of chemotherapy-induced neutropenia and risk of infection among patients with non-myeloid malignancies. Support Care Cancer 2016; 24(10): 4377-83; Caggiano et al., Incidence, cost, and mortality of neutropenia hospitalization associated with chemotherapy. Cancer. 2005; 103(9): 1916-24). Moreover, CIM commonly leads to dose reductions and delays, which limit therapeutic dose intensity and can compromise the anti-tumor efficacy benefits of chemotherapy. In some instances, treatment is discontinued. For example, a 25% dose reduction is recommended on the first instance, and a 50% dose reduction is recommended on the second instance, and discontinuation is recommended on the third instance of any of Grade 4 neutropenia >7 days, Grade 3 febrile neutropenia (ANC <1000/mm 3 and fever ≥38.5° C.), or at anytime of scheduled treatment, Grade 3-4 neutropenia which delays dosing by 2 or 3 weeks for recovery to ≤Grade 1. Treatment discontinuation is recommended upon the first instance of Grade 3-4 neutropenia which delays dosing beyond 3 weeks for recovery to ≤Grade 1.

Attempts at developing and implementing clinical algorithms to guide chemotherapy dose reductions and treatment delays in patients with neutropenia and/or thrombocytopenia during treatments have been examined (see, for example, Clinical Trial of a Novel Dose Adjustment Algorithm for Preventing Cytopenia-Related Delays During FOLFOX Chemotherapy, ClinicalTrials.gov Identifier: NCT04526886). Nonetheless, chemotherapy-induced cellular damage to the immune system may also limit anti-tumor efficacy due to an inability of the host immune system to effectively mount a response against the cancer. Prolonged exposure to myelosuppressive agents can lead to cumulative bone marrow toxicity and myelosuppression that can limit the ability to deliver subsequent lines of therapy at the standard of care doses and schedule. To date, there is no single treatment available that prevents or mitigates the myelosuppressive effects of sacituzumab govitecan chemotherapy protocols before they occur. Existing interventions are generally used reactively to treat acute cytopenias and are lineage specific; each of these have their own set of associated risks (see, e.g., Blumberg et al., (2010) Platelet transfusions: trigger, dose, benefits, and risks. F1000 Med Rep 2:5. https://doi.org/10.3410/m2-5; Bohlius et al., (2019) Management of cancer-associated anemia with erythropoiesis-stimulating agents: ASCO/ASH Clinical Practice Guideline Update. J Clin Oncol 37 (15):1336-1351.https://doi.org/10.1200/jco.18.02142; Xu et al., (2016) Risk factors for bone pain among patients with cancer receiving myelosuppressive chemotherapy and pegfilgrastim. Support Care Cancer 24 (2):723-730. https://doi.org/10.1007/s00520-015-2834-2; Corey-Lisle et al., (2014) Transfusions and patient burden in chemotherapy-induced anaemia in France. Ther Adv Med Oncol 6 (4):146-153. https://doi.org/10.1177/1758834014534515).

The development of chemotherapy-induced diarrhea (CID) can be debilitating and, in some cases, life threatening. Findings in such patients include volume depletion, renal failure, and electrolyte disorders such as metabolic acidosis and depending upon water intake, hyponatremia (increased water intake that cannot be excreted because of the hypovolemic stimulus to the release of antidiuretic hormone) or hypernatremia (insufficient water intake to replace losses) (Maroun et al., (2007) Prevention and management of chemotherapy-induced diarrhea in patients with colorectal cancer: a consensus statement by the Canadian working group on chemotherapy-induced diarrhea. Curr Oncol 14: 13-20). CID can interfere with and detract from cancer treatment by causing dosing delays or reductions which may have an impact on survival. For example, a 25% dose reduction is recommended on the first instance, a 50% dose reduction is recommended on the second instance, and discontinuation is recommended on the third instance of Grade 3-4 diarrhea that is not controlled with anti-emetic and anti-diarrheal agents.

Stomatitis/mucositis is a result of the toxic effect of chemotherapeutics on the rapidly dividing epithelial cells lining the gastro-intestinal tract (which goes from the mouth to the anus), leaving the mucosal tissue open to ulceration and infection. Stomatitis/mucositis generally begins 5-10 days following the initiation of chemotherapy and lasts anywhere from one week to six weeks or more. Many patients with stomatitis/mucositis develop significant nutritional issues due to the inability to eat because of the associated pain, leading to hypovolemia, electrolyte abnormalities, malnutrition, and even death. Severe stomatitis/mucositis often results in dose reductions of or interruptions to the therapeutic protocol. For example, a 25% dose reduction is recommended on the first instance, a 50% dose reduction is recommended on the second instance, and discontinuation is recommended on the third instance of Grade 4 mucositis or stomatitis, or Grade 3-4 mucositis or stomatitis persisting >48 hours despite optimal medical management, or at time of scheduled treatment, Grade 3-4 mucositis or stomatitis which delays dosing by 2 or 3 weeks for recovery to <Grade 1. Discontinuation of dosing is recommended on the first instance of Grade 3-4 mucositis or stomatitis which does not recover to <Grade 1 within 3 weeks.

Sacituzumab govitecan is generally administered by intravenous infusion on day 1 and day 8 of each 21-day cycle after administration of trilaciclib. As provided herein, administration of sacituzumab govitecan should not be longer than 3 hours. In some embodiments, the infusion is administered over 3 hours. In some embodiments, the infusion is administered over 2 hours. In some embodiments, the infusion is administered over 1 hour. In the methods provided herein, sacituzumab govitecan can be administered according to institutional guidelines. In some embodiments, sacituzumab govitecan can be administered at its standard of care dose of 10 mg/kg. In some embodiments, sacituzumab govitecan is administered at a dose of between about 5 mg/kg and 15 mg/kg. In some embodiments, sacituzumab govitecan is administered at a dose of about 5 mg/kg. In some embodiments, sacituzumab govitecan is administered at a dose of about 6 mg/kg. In some embodiments, sacituzumab govitecan is administered at a dose of about 7 mg/kg. In some embodiments, sacituzumab govitecan is administered at a dose of about 8 mg/kg. In some embodiments, sacituzumab govitecan is administered at a dose of about 9 mg/kg. In some embodiments, sacituzumab govitecan is administered at a dose of about 10 mg/kg.

In some embodiments, prior to the administration of sacituzumab govitecan, the patient is premedicated for the prevention of infusion reactions and chemotherapy-induced nausea and vomiting (CINV). In some embodiments, prior to the administration of sacituzumab govitecan-hziy, the patient is premedicated with antipyretics, histamine receptor 1 (H1) and histamine receptor 2 (H2) blockers, and corticosteroids for the prevention of infusion reactions. In some embodiments, prior to the administration of sacituzumab govitecan-hziy, the patient is premedicated with dexamethasone in combination with either a serotonin 5-HT3 receptor antagonist or a Neurokinin 1 (NK1) receptor antagonist for the prevention of chemotherapy-induced nausea and vomiting (CINV).

### Advanced/Metastatic Treatment of TNBC Using Trilaciclib+Sacituzumab Govitecan

As provided herein, the defined subpopulations of patients with advanced/metastatic TNBC as described herein are administered trilaciclib, or a pharmaceutically acceptable salt thereof, in combination with the antibody drug conjugate sacituzumab govitecan in a specifically timed administrative protocol. Accordingly, provided herein is a method of treating a human patient with advanced/metastatic TNBC comprising:
i) administering to the patient an effective amount of a CDK 4/6 inhibitor having the structure:

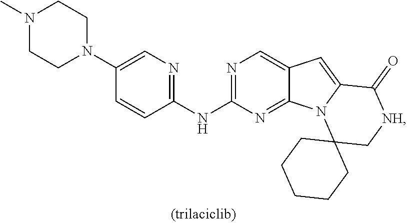 [SMILES: CN1CCN(c2ccc(Nc3ncc4cc5n(c4n3)C3(CCCCC3)CNC5=O)nc2)CC1]

or a pharmaceutically acceptable salt, composition, isotope, or prodrug thereof;
ii) administering to the patient an effective amount of sacituzumab govitecan, wherein trilaciclib is administered prior to the initiation of administration of sacituzumab govitecan, and wherein the patient has received at least two prior treatments with at least one in the metastatic setting.

In some embodiments, trilaciclib is administered less than 4 hours or prior to the administration of sacituzumab govitecan. In some embodiments, trilaciclib is administered about 2 hour or less, for example, about 2 hours, about 1 hour and 30 minutes, about 1 hour, about 45 minutes, about 40 minutes, about 35 minutes, or about 30 minutes, prior to administration of sacituzumab govitecan.

In some embodiments, trilaciclib is administered to the patient intravenously between about 190 and 280 mg/m². In some embodiments, the trilaciclib is administered at about 240 mg/m².

In some embodiments, sacituzumab govitecan is administered at a dose of between about 5 mg/kg and 15 mg/kg. In some embodiments, sacituzumab govitecan is administered at a dose of about 5 mg/kg. In some embodiments, sacituzumab govitecan is administered at a dose of about 6 mg/kg. In some embodiments, sacituzumab govitecan is administered at a dose of about 7 mg/kg. In some embodiments, sacituzumab govitecan is administered at a dose of about 8 mg/kg. In some embodiments, sacituzumab govitecan is administered at a dose of about 9 mg/kg. In some embodiments, sacituzumab govitecan is administered at a dose of about 10 mg/kg. In some embodiments, sacituzumab govitecan is administered as a continuous infusion (CI) over a period of between about 1 hour to 3 hours. In some embodiments, the first infusion of sacituzumab govitecan is administered over 3 hours. In some embodiments, subsequent infusions of sacituzumab govitecan is administered over 2 hours. In some embodiments, subsequent infusions of sacituzumab govitecan is administered over 1 hour.

In some embodiments, the trilaciclib/sacituzumab govitecan regimen is administered in 1 or more cycles, 2 or more cycles, 3 or more cycles, 4 or more cycles, 5 or more cycles, 6 or more cycles, 7 or more cycles, 8 or more cycles, 9 or more cycles, 10 or more cycles, 11 or more cycles, 12 or more cycles, 13 or more cycles, 14 or more cycles, 15 or more cycles, 16 or more cycles, 17 or more cycles, 18 or more cycles, 19 or more cycles, 20 or more cycles, 21 or more cycles, 22 or more cycles, 23 or more cycles, 24 or more cycles, 25 or more cycles, 26 or more cycles, 27 or more cycles, 28 or more cycles, 29 or more cycles, 30 or more cycles, 31 or more cycles, 32 or more cycles, 33 or more cycles, or 34 or more cycles. In some embodiments, the trilaciclib/sacituzumab govitecan regimen is administered up to 35 times.

In some embodiments, the protocol comprises one or more 21-day therapeutic cycles, wherein trilaciclib and sacituzumab govitecan are administered on days 1 and 8 of each 21-day cycle, wherein trilaciclib is administered no more than 4 hours prior to the administration of the sacituzumab govitecan-, and wherein the trilaciclib is completely administered before the start of the administration of sacituzumab govitecan.

In some embodiments, the protocol comprises one or more 21-day therapeutic cycles, wherein trilaciclib and sacituzumab govitecan are administered on days 1 and 8 of each 21-day cycle, and trilaciclib without sacituzumab govitecan is administered on day 15 of each 21-day cycle, wherein trilaciclib is administered no more than 4 hours prior to the administration of the sacituzumab govitecan-, and wherein the trilaciclib is completely administered before the start of the administration of sacituzumab govitecan on day 1 and day 8.

In some embodiments, prior to the administration of sacituzumab govitecan, the patient is premedicated for the prevention of infusion reactions and chemotherapy-induced nausea and vomiting (CINV). In some embodiments, prior to the administration of sacituzumab govitecan, the patient is premedicated with antipyretics, histamine receptor 1 (H1) and histamine receptor 2 (H2) blockers, and corticosteroids for the prevention of infusion reactions. In some embodiments, prior to the administration of sacituzumab govitecan, the patient is premedicated with dexamethasone in combination with either a serotonin 5-HT3 receptor antagonist or a Neurokinin 1 (NK1) receptor antagonist for the prevention of chemotherapy-induced vomiting and nausea (CINV).

### Advanced/Metastatic Treatment of Urothelial Cancer Using Trilaciclib+Sacituzumab Govitecan

As provided herein, the defined subpopulations of patients with advanced/metastatic urothelial cancer as described herein are administered trilaciclib, or a pharmaceutically acceptable salt thereof, in combination with the antibody drug conjugate sacituzumab govitecan in a specifically timed administrative protocol. Accordingly, provided herein is a method of treating a human patient with advanced/metastatic urothelial cancer comprising:
i) administering to the patient an effective amount of a CDK 4/6 inhibitor having the structure:

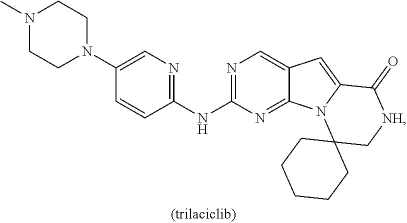 [SMILES: CN1CCN(c2ccc(Nc3ncc4cc5n(c4n3)C3(CCCCC3)CNC5=O)nc2)CC1]

or a pharmaceutically acceptable salt, composition, isotope, or prodrug thereof;
ii) administering to the patient an effective amount of sacituzumab govitecan;
wherein trilaciclib is administered prior to the initiation of administration of sacituzumab govitecan, and wherein the patient has received a platinum-containing chemotherapy and either programmed death receptor-1 (PD-1) or programmed death ligand-1 (PD-L1) inhibitor.

In some embodiments, trilaciclib is administered less than 4 hours or prior to the administration of sacituzumab govitecan. In some embodiments, trilaciclib is administered about 2 hour or less, for example, about 2 hours, about 1 hour and 30 minutes, about 1 hour, about 45 minutes, about 40 minutes, about 35 minutes, or about 30 minutes, prior to administration of sacituzumab govitecan. In some embodiments, trilaciclib is administered to the patient intravenously between about 190 and 280 mg/m². In some embodiments, the trilaciclib is administered at about 240 mg/m².

In some embodiments, sacituzumab govitecan is administered at a dose of between about 5 mg/kg and 15 mg/kg. In some embodiments, sacituzumab govitecan is administered at a dose of about 5 mg/kg. In some embodiments, sacituzumab govitecan is administered at a dose of about 6 mg/kg. In some embodiments, sacituzumab govitecan is administered at a dose of about 7 mg/kg.

In some embodiments, sacituzumab govitecan is administered at a dose of about 8 mg/kg. In some embodiments, sacituzumab govitecan is administered at a dose of about 9 mg/kg. In some embodiments, sacituzumab govitecan is administered at a dose of about 10 mg/kg. In some embodiments, sacituzumab govitecan is administered as a continuous infusion (CI) over a period of between about 1 hour to 3 hours. In some embodiments, the first infusion of sacituzumab govitecan is administered over 3 hours. In some embodiments, subsequent infusions of sacituzumab govitecan is administered over 2 hours. In some embodiments, subsequent infusions of sacituzumab govitecan is administered over 1 hour.

In some embodiments, the trilaciclib/sacituzumab govitecan regimen is administered in 1 or more cycles, 2 or more cycles, 3 or more cycles, 4 or more cycles, 5 or more cycles, 6 or more cycles, 7 or more cycles, 8 or more cycles, 9 or more cycles, 10 or more cycles, 11 or more cycles, 12 or more cycles, 13 or more cycles, 14 or more cycles, 15 or more cycles, 16 or more cycles, 17 or more cycles, 18 or more cycles, 19 or more cycles, 20 or more cycles, 21 or more cycles, 22 or more cycles, 23 or more cycles, 24 or more cycles, 25 or more cycles, 26 or more cycles, 27 or more cycles, 28 or more cycles, 29 or more cycles, 30 or more cycles, 31 or more cycles, 32 or more cycles, 33 or more cycles, or 34 or more cycles. In some embodiments, the trilaciclib/sacituzumab govitecan regimen is administered up to 35 times.

In some embodiments, prior to the administration of sacituzumab govitecan, the patient is premedicated for the prevention of infusion reactions and chemotherapy-induced nausea and vomiting (CINV). In some embodiments, prior to the administration of sacituzumab govitecan, the patient is premedicated with antipyretics, histamine receptor 1 (H1) and histamine receptor 2 (H2) blockers, and corticosteroids for the prevention of infusion reactions. In some embodiments, prior to the administration of sacituzumab govitecan, the patient is premedicated with dexamethasone in combination with either a serotonin 5-HT3 receptor antagonist or a Neurokinin 1 (NK1) receptor antagonist for the prevention of chemotherapy-induced nausea and vomiting (CINV).

In some embodiments, the protocol comprises one or more 21-day therapeutic cycles, wherein trilaciclib and sacituzumab govitecan are administered on days 1 and 8 of each 21-day cycle, wherein trilaciclib is administered no more than 4 hours prior to the administration of the sacituzumab govitecan, and wherein the trilaciclib is completely administered before the start of the administration of sacituzumab govitecan.

In some embodiments, the protocol comprises one or more 21-day therapeutic cycles, wherein trilaciclib and sacituzumab govitecan are administered on days 1 and 8 of each 21-day cycle, and wherein trilaciclib is administered without sacituzumab govitecan on day 15 of each 21 day cycle, wherein trilaciclib is administered no more than 4 hours prior to the administration of the sacituzumab govitecan on day 1 and day 8, and wherein the trilaciclib is completely administered before the start of the administration of sacituzumab govitecan.

Treatment of Trop-2 Overexpressing Cancer with Trilaciclib+Sacituzumab govitecan

As provided herein, the defined subpopulations of patients with a Trop-2 overexpressing cancer as described herein are administered trilaciclib, or a pharmaceutically acceptable salt thereof, in combination with the antibody drug conjugate sacituzumab govitecan in a specifically timed administrative protocol. Accordingly, provided herein is a method of treating a human patient with a Trop-2 overexpressing cancer comprising:
i) administering to the patient an effective amount of a CDK 4/6 inhibitor having the structure:

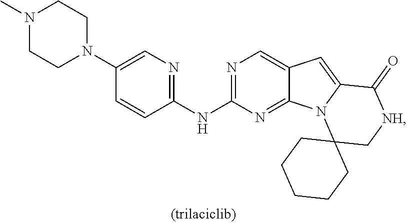 [SMILES: CN1CCN(c2ccc(Nc3ncc4cc5n(c4n3)C3(CCCCC3)CNC5=O)nc2)CC1]

or a pharmaceutically acceptable salt, composition, isotope, or prodrug thereof; ii) administering to the patient an effective amount of sacituzumab govitecan; wherein trilaciclib is administered prior to the initiation of administration of sacituzumab govitecan.

In some embodiments, the Trop-2 overexpressing cancer is non-small cell lung cancer (NSCLC). In a particular embodiment, the NSCLC is metastatic or advanced NSCLC. In some embodiments, the NSCLC has progressed on or after platinum-based chemotherapy and PD-1 or PD-L1 inhibitor therapy received either in combination or sequentially.

In some embodiments, trilaciclib is administered less than 4 hours or prior to the administration of sacituzumab govitecan. In some embodiments, trilaciclib is administered about 2 hour or less, for example, about 2 hours, about 1 hour and 30 minutes, about 1 hour, about 45 minutes, about 40 minutes, about 35 minutes, or about 30 minutes, prior to administration of sacituzumab govitecan. In some embodiments, trilaciclib is administered to the patient intravenously between about 190 and 280 mg/m². In some embodiments, the trilaciclib is administered at about 240 mg/m².

In some embodiments, sacituzumab govitecan is administered at a dose of between about 5 mg/kg and 15 mg/kg. In some embodiments, sacituzumab govitecan is administered at a dose of about 5 mg/kg. In some embodiments, sacituzumab govitecan is administered at a dose of about 6 mg/kg. In some embodiments, sacituzumab govitecan is administered at a dose of about 7 mg/kg. In some embodiments, sacituzumab govitecan is administered at a dose of about 8 mg/kg. In some embodiments, sacituzumab govitecan is administered at a dose of about 9 mg/kg. In some embodiments, sacituzumab govitecan is administered at a dose of about 10 mg/kg. In some embodiments, sacituzumab govitecan is administered as a continuous infusion (CI) over a period of between about 1 hour to 3 hours. In some embodiments, the first infusion of sacituzumab govitecan is administered over 3 hours. In some embodiments, subsequent infusions of sacituzumab govitecan is administered over 2 hours. In some embodiments, subsequent infusions of sacituzumab govitecan is administered over 1 hour.

In some embodiments, the trilaciclib/sacituzumab govitecan regimen is administered in 1 or more cycles, 2 or more cycles, 3 or more cycles, 4 or more cycles, 5 or more cycles, 6 or more cycles, 7 or more cycles, 8 or more cycles, 9 or more cycles, 10 or more cycles, 11 or more cycles, 12 or more cycles, 13 or more cycles, 14 or more cycles, 15 or more cycles, 16 or more cycles, 17 or more cycles, 18 or more cycles, 19 or more cycles, 20 or more cycles, 21 or more cycles, 22 or more cycles, 23 or more cycles, 24 or more cycles, 25 or more cycles, 26 or more cycles, 27 or more cycles, 28 or more cycles, 29 or more cycles, 30 or more cycles, 31 or more cycles, 32 or more cycles, 33 or more cycles, or 34 or more cycles. In some embodiments, the trilaciclib/sacituzumab govitecan regimen is administered up to 35 times.

In some embodiments, prior to the administration of sacituzumab govitecan, the patient is premedicated for the prevention of infusion reactions and chemotherapy-induced nausea and vomiting (CINV). In some embodiments, prior to the administration of sacituzumab govitecan, the patient is premedicated with antipyretics, histamine receptor 1 (H1) and histamine receptor 2 (H2) blockers, and corticosteroids for the prevention of infusion reactions. In some embodiments, prior to the administration of sacituzumab govitecan, the patient is premedicated with dexamethasone in combination with either a serotonin 5-HT3 receptor antagonist or a Neurokinin 1 (NK1) receptor antagonist for the prevention of chemotherapy-induced nausea and vomiting (CINV).

In some embodiments, the protocol comprises one or more 21-day therapeutic cycles, wherein trilaciclib and sacituzumab govitecan are administered on days 1 and 8 of each 21-day cycle, wherein trilaciclib is administered no more than 4 hours prior to the administration of the sacituzumab govitecan, and wherein the trilaciclib is completely administered before the start of the administration of sacituzumab govitecan.

Trop-2 overexpressing cancer for treatment with the methods described herein include advanced/metastatic cancers selected from the group consisting of breast cancer, including TNBC, cervical cancer, colon or colorectal cancer, endometroid endometrial cancer, esophageal cancer, gastric cancer, gliomas, hilar cholangiocarcinoma, squamous cell carcinoma of the oral cavity, gastrointestinal cancer, chronic lymphocytic lymphoma, extranodal NK/T-cell lymphoma, non-Hodgkin's lymphoma, Raji Burkitt lymphoma, small-sized pulmonary adenocarcinoma, ovarian cancer, pancreatic cancer, prostate cancer, stomach carcinoma, thyroid carcinoma, urothelial carcinoma, uterine cancer, and lung cancer, including small cell lung cancer and non-small cell lung cancer. In some embodiments, the Trop-2 overexpressing cancer is breast cancer. In some embodiments, the Trop-2 overexpressing cancer is triple negative breast cancer. In some embodiments, the Trop-2 overexpressing cancer is urothelial cancer. In some embodiments, the Trop-2 overexpressing cancer is colon or colorectal cancer. In some embodiments, the Trop-2 overexpressing cancer is prostate cancer. In some embodiments, the Trop-2 overexpressing cancer is pancreatic cancer. In some embodiments, the Trop-2 overexpressing cancer is lung cancer. In some embodiments, the Trop-2 overexpressing lung cancer is non-small cell lung cancer. In some embodiments, the Trop-2 overexpressing lung cancer is small cell lung cancer. In some embodiments, the Trop-2 overexpressing cancer is cervical cancer. In some embodiments, the Trop-2 overexpressing cancer is endometroid endometrial cancer. In some embodiments, the Trop-2 overexpressing cancer is esophageal cancer. In some embodiments, the Trop-2 overexpressing cancer is gastric cancer. In some embodiments, the Trop-2 overexpressing cancer is a glioma. In some embodiments, the Trop-2 overexpressing cancer is hilar cholangiocarcinoma. In some embodiments, the Trop-2 overexpressing cancer is squamous cell carcinoma of the oral cavity. In some embodiments, the Trop-2 overexpressing cancer is gastrointestinal cancer. In some embodiments, the Trop-2 overexpressing cancer is chronic lymphocytic lymphoma. In some embodiments, the Trop-2 overexpressing cancer is extranodal NK/T-cell lymphoma. In some embodiments, the Trop-2 overexpressing cancer is non-Hodgkin's lymphoma. In some embodiments, the Trop-2 overexpressing cancer is Raji Burkitt lymphoma. In some embodiments, the Trop-2 overexpressing cancer is small-sized pulmonary adenocarcinoma. In some embodiments, the Trop-2 overexpressing cancer is ovarian cancer. In some embodiments, the Trop-2 overexpressing cancer is pancreatic cancer. In some embodiments, the Trop-2 overexpressing cancer is prostate cancer. In some embodiments, the Trop-2 overexpressing cancer is stomach carcinoma. In some embodiments, the Trop-2 overexpressing cancer is thyroid carcinoma. In some embodiments, the Trop-2 overexpressing cancer is uterine cancer.

Trophoblast cell surface antigen 2 (Trop-2) is a glycoprotein that spans the epithelial membrane surface and plays a role in cell self-renewal, proliferation, and transformation (Zaman et al., Targeting Trop-2 in solid tumors: future prospects. Onco Targets Ther. 2019; 12: 1781-1790). Under physiological conditions, Trop-2 plays an essential role in embryonic development, placental tissue formation, embryo implantation, stem cell proliferation, and organ development (Shvartsur et al., Trop2 and its overexpression in cancers: regulation and clinical/therapeutic implications. Genes Cancer. 2015; 6(3-4): 84-105). A low basal expression level of Trop-2 is found on the surface of multiple normal epithelial tissues, including skin and oral mucosa (Strop P, Tran T T, Dorywalska M, et al. RN927C, a site-specific Trop-2 antibody-drug conjugate (ADC) with enhanced stability, is highly efficacious in preclinical solid tumor models. Mol Cancer Ther. 2016; 15(11): 2698-2708). Trop-2 can promote tumor growth and its overexpression is common in many types of malignant epithelial tumors (Goldenberg D M, Stein R, Sharkey R M. The emergence of trophoblast cell-surface antigen 2 (TROP-2) as a novel cancer target. Oncotarget. 2018; 9(48): 28989-29006). Overexpression of Trop-2 accelerates the cancer cell cycle and drives cancer growth. Trop2 overexpression is associated with decreased patient survival as well as increased tumor aggressiveness and metastasis in many cancers. In some embodiments, patients with advanced/metastatic cancers that overexpress Trop-2 are administered trilaciclib, or a pharmaceutically acceptable salt thereof, in combination with the antibody drug conjugate sacituzumab govitecan in a specifically timed administrative protocol described herein. In some embodiments, the advanced/metastatic cancers that overexpress Trop-2 are selected from the group consisting of breast cancer, cervical cancer, colon and colorectal cancer, endometroid endometrial cancer, esophageal cancer, gastric cancer, gliomas, hilar cholangiocarcinoma, squamous cell carcinoma of the oral cavity, gastrointestinal cancer, chronic lymphocytic lymphoma, extranodal NK/T-cell lymphoma, non-Hodgkin's lymphoma, Raji Burkitt lymphoma, small-sized pulmonary adenocarcinoma, ovarian cancer, pancreatic cancer, prostate cancer, stomach carcinoma, thyroid carcinoma, urinary bladder carcinoma, and uterine cancer.

Methods of measuring Trop-2 expression are known in the art. One method is to measure the presence of the TROP2 gene from tumor sample mRNA with expressed sequence tag (EST) analysis, serial analysis of gene expression (SAGE), DNA microarray analysis and/or quantitative RT-PCR as well as directly from tumor samples using immunohistochemistry methods. These methods are described in Trerotola, M., Cantanelli, P., Guerra, E. et al. Upregulation of Trop-2 quantitatively stimulates human cancer growth. Oncogene 32, 222-233 (2013). Trop-2 expression can also be measured using flow cytometry techniques as described in Zeybek B, Manzano A, Bianchi A, et al. Cervical carcinomas that overexpress human trophoblast cell-surface marker (Trop-2) are highly sensitive to the antibody-drug conjugate sacituzumab govitecan. Sci Rep. 2020; 10(1): 973. Published 2020 Jan. 22. Immunofluorescent techniques can also be used to measure Trop-2 expression as described in Strop et al., N927C, a Site-Specific Trop-2 Antibody— Drug Conjugate (ADC) with Enhanced Stability, Is Highly Efficacious in Preclinical Solid Tumor Models; Mol Cancer Ther Nov. 1, 2016 (15) (11) 2698-2708.

### Anti-Tumor Efficacy Assessment

In some embodiments, the inclusion of trilaciclib in a sacituzumab govitecan chemotherapy protocol described herein for the treatment of advanced or metastatic TNBC or metastatic urothelial cancer, or other Trop-2 overexpressing cancer such as NSCLC, provides for an increased anti-tumor efficacy compared to those patients receiving a sacituzumab govitecan chemotherapy protocol without trilaciclib. Methods of accessing tumor response are well known in the art and include, for example RECIST v1.1 (Eisenhauer et al., New response evaluation criteria in solid tumors: revised RECIST guideline (version 1.1). Eur J Cancer. 2009; 45: 228-247).

In some embodiments, the inclusion of trilaciclib in a 58acituzumab govitecan chemotherapy protocol described herein for the treatment of advanced or metastatic TNBC, alternatively advanced or metastatic urothelial cancer, or still alternatively an advanced or metastatic Trop-2 overexpressing cancer, for example but not limited to non-small cell lung cancer provides for an increased or extended progression free survival (PFS) compared to those patients not receiving trilaciclib. PFS is generally defined as the time (number of months) from date of initial administration of trilaciclib+sacituzumab govitecan as provided herein until the date of documented radiologic disease progression (PD) per RECIST v1.1 or death due to any cause, whichever comes first. Methods of accessing increased PFS are well known in the art and include, for example RECIST v1.1 (Eisenhauer et al., New response evaluation criteria in solid tumors: revised RECIST guideline (version 1.1). Eur J Cancer. 2009; 45: 228-247).

In some embodiments, the inclusion of trilaciclib in a sacituzumab govitecan chemotherapy protocol described herein for the treatment of advanced or metastatic TNBC or alternatively advanced or metastatic urothelial cancer, or other Trop-2 overexpressing cancer including but not limited to NSCLC provides for an increased or extended overall survival (OS) compared to those patients not receiving trilaciclib. OS is generally calculated as the time (months) from the date of initial administration of trilaciclib+sacituzumab govitecan as provided herein to the date of death for patients who died due to any cause compared to patients not receiving trilaciclib.

In some embodiments, the inclusion of trilaciclib in a sacituzumab govitecan chemotherapy protocol described herein for the treatment of advanced or metastatic TNBC, alternatively advanced or metastatic urothelial cancer, or still alternatively a Trop-2 overexpressing cancer including but not limited to NSCLC provides for an improved objective response rate (ORR) compared to those patients not receiving trilaciclib. ORR is generally defined as the proportion of patients with best overall response (BOR) of either complete response (CR) or partial response (PR) per RECIST v1.1. Examples of an objective response (OR) includes a complete response (CR), which is the disappearance of all signs of the tumor in response to treatment and a partial response (PR), which is a decrease in the size of a tumor in response to treatment. In some embodiments, the objective response (OR) is a complete response (CR). In some embodiments, the objective response (OR) is a partial response (PR). The ORR is an important parameter to demonstrate the efficacy of a treatment and it serves as a primary or secondary end-point in clinical trials. Methods of assessing ORR are well known in the art and include, for example RECIST v1.1 (Eisenhauer et al., New response evaluation criteria in solid tumors: revised RECIST guideline (version 1.1). Eur J Cancer. 2009; 45: 228-247) and World Health Organization (WHO) (World Health Organization. WHO Handbook for Reporting Results of Cancer Treatment. World Health Organization Offset Publication No. 48; Geneva (Switzerland), 1979). Statistical methods of measuring objective response rate are well known in the art and include, for example, the Clopper-Pearson Method (Clopper, C.; Pearson, E. S. (1934). “The use of confidence or fiducial limits illustrated in the case of the binomial”. Biometrika. 26 (4): 404-413. doi:10.1093/biomet/26.4.404).

In some embodiments, the inclusion of trilaciclib in a sacituzumab govitecan chemotherapy protocol described herein for the treatment of advanced or metastatic TNBC, alternatively advanced or metastatic urothelial cancer, or still alternatively a Trop-2 overexpressing cancer including but not limited to NSCLC provides for an improved clinical benefit rate (CBR) compared to those patients not receiving trilaciclib. CBR is generally defined as the proportion of patients with a best overall response of complete response (CR), partial response (PR) or stable disease (SD) lasting 24 weeks or longer since the first date of study administration. Methods of assessing improved CBR are well known in the art and include, for example RECIST v1.1 (Eisenhauer et al., New response evaluation criteria in solid tumors: revised RECIST guideline (version 1.1). Eur J Cancer. 2009; 45: 228-247).

In some embodiments, the inclusion of trilaciclib in a sacituzumab govitecan chemotherapy protocol described herein for the treatment of advanced or metastatic TNBC, alternatively advanced or metastatic urothelial cancer, or still alternatively a Trop-2 overexpressing cancer including but not limited to NSCLC provides for an improved Duration of Objective Response (DOR) compared to those patients not receiving trilaciclib. DOR is generally defined as the time (months) between the date of achieving first objective response (CR or PR), confirmed at the next tumor scan, and the date of documented disease progression per RECIST v1.1 or death, whichever comes first. Methods of assessing improved DOR are well known in the art and include, for example RECIST v1.1 (Eisenhauer et al., New response evaluation criteria in solid tumors: revised RECIST guideline (version 1.1). Eur J Cancer. 2009; 45: 228-247).

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC provides for T-cell immune activation against the tumor. In some embodiments, the T-cell immune activation results in T-cell receptor (TCR) modulation. In some embodiments, the T-cell activation results in increased interferon gamma (IFNγ) expression. In some embodiments, the T-cell activation results in increased activation-induced expression of CD137. In some embodiments, the T-cell activation results in increased TCR diversity.

In one aspect, the improved methods of treatment are administered to a select patient subgroup with advanced/metastatic TNBC, or alternatively advanced/metastatic urothelial carcinoma, or still alternatively a Trop-2 overexpressing cancer including but not limited to NSCLC with a threshold Simpson clonality score. Simpson clonality is a T-cell clonal diversity single number metric that describes the characteristic shape of a sample repertoire. Simpson clonality measures the evenness of the repertoire, that is the extent to which one or a few clones dominate the sample repertoire. Simpson Clonality is calculated as follows:

$$SIMPSON CLONALITY = \sqrt{{\sum }_{i = 1}^{R} {P}_{i}^{2}} ,$$

wherein R=the total number of rearrangements; i=each rearrangement; Pi=productive frequency of rearrangement i. Simpson Clonality is the square root of Simpson's Index. Simpson's Index is 1—Simpson's Diversity Index. Simpson clonality is further described in, for example, Wong, et al. J Immunol. 2016; 197(5): 1642-9; Schneider-Hohendorf, et al. Nat Commun. 2016; 7: 11153; Weir, et al. J Immunother Cancer. 2016; 4: 68; Nunes-Alves, et al. PLoS Pathog. 2015; 11(5): e1004849; Suessmuth, et al. Blood. 2015; 125(25): 3835-50; Mahalingam, et al. Clin Can Res. 2020; 26(1): 71-81; Morris, et al. Sci Transl Med. 2015; 7(272): 272ra10; Roh, et al. Sci Transl Med. 2017; 9(379); Zhu, et al. Oncolmmunology. 2015; 4(12): e1051922; Tumeh, et al. Nature. 2014;515(7528):568-71; Keane, et al. Clin Cancer Res. 2017;23(7):1820-1828; Kirsch, et al. Sci Transl Med. 2015; 7(308): 308ra158; Hershberg, et al. Phil. Trans. R. Soc. B. 2015; 370(1676)20140239; Wu, et al. Sci Transl Med. 2012; 4(134) L 134ra63; Carey, et al. J Immunol. 2016; 196(6):2602-13; Seay, et al. JCI Insight. 2016; 1(20): e88242; Emerson, et al. Nat Genet. 2017; 49(5): 659-665; Lindau et al. J Immunol. 2018; ji1800217, each of which is incorporated herein by reference. 0 represents a completely even sample, while 1 represents a monoclonal sample.

In some embodiments, the administration of the trilaciclib/sacituzumab govitecan regimen results in a decrease from a baseline Simpson clonality, wherein the baseline is measured at the start of trilaciclib/sacituzumab govitecan.

### Reduction of Toxicity

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in improved myelopreservation of hematopoietic stem and progenitor cells (HSPCs) and/or immune effector cells such as lymphocytes including T-lymphocytes, as well as enhanced anti-tumor efficacy in patients compared to those receiving a sacituzumab govitecan chemotherapy protocol without trilaciclib. Improvements in myelopreservation of hematopoietic stem and progenitor cells (HSPCs) and immune effector cells such as lymphocytes including T-lymphocytes are measured with increases in hematology assessments (complete blood count (CBC), red blood cell count (RBC), platelet count, white blood cell count (WBC) and absolute neutrophil count (ANC)), reduction in severe adverse events (AEs), reduction in supportive care interventions (including transfusions and G-CSF administration), reduction in dose modifications, and improved patient recorded outcomes (PROs).

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in a reduction in the incidence of chemotherapeutic induced-myelosuppression (CIM). Patients who develop myelosuppression while receiving a chemotherapeutic agent such as sacituzumab govitecan are more likely to experience infections, sepsis, bleeding, and fatigue, often leading to the need for hospitalizations, hematopoietic growth factor support, transfusions (red blood cells [RBCs] and/or platelets), and even death (see, e.g., Gustinetti et al., Bloodstream infections in neutropenic cancer patients: A practical update. Virulence. 2016; 7(3): 280-97; Li et al., Relationship between severity and duration of chemotherapy-induced neutropenia and risk of infection among patients with nonmyeloid malignancies. Support Care Cancer 2016; 24(10): 4377-83; Caggiano et al., Incidence, cost, and mortality of neutropenia hospitalization associated with chemotherapy. Cancer. 2005; 103(9): 1916-24). Moreover, CIM commonly leads to dose reductions and delays, which limit therapeutic dose intensity and can compromise the anti-tumor efficacy benefits of chemotherapy. Attempts at developing and implementing clinical algorithms to guide chemotherapy dose reductions and treatment delays in patients with neutropenia and/or thrombocytopenia during treatments have been examined (see, for example, Clinical Trial of a Novel Dose Adjustment Algorithm for Preventing Cytopenia-Related Delays During FOLFOX Chemotherapy, ClinicalTrials.gov Identifier: NCT04526886). Nonetheless, chemotherapy-induced cellular damage to the immune system may also limit anti-tumor efficacy due to an inability of the host immune system to effectively mount a response against the cancer. Prolonged exposure to myelosuppressive agents can lead to cumulative bone marrow toxicity and myelosuppression that can limit the ability to deliver subsequent lines of therapy at the standard of care doses and schedule.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in myelopreservation of the neutrophil lineage in patients compared to those receiving the sacituzumab govitecan chemotherapy protocol without trilaciclib. Endpoints to measure myelopreservation of the neutrophil lineage include a reduction in duration of severe neutropenia, for example after cycle 1, and the reduction in the occurrence of severe neutropenia. Neutropenia is generally defined as a condition that results when the body does not have enough neutrophils, an important white blood cell that fights infections. The lower the neutrophil count, the more vulnerable one is to infectious diseases. Neutropenia is generally quantified as an absolute neutrophil count (ANC) of less than 1500 per microliter (1500/μL). Severe neutropenia is generally quantified as an absolute neutrophil count (ANC) of less than 500 per microliter (500/μL). Methods of calculating absolute neutrophil count (ANC) are well known in the art and include multiplying the WBC count times the percent of neutrophils in the differential WBC count. The percent of neutrophils consists of the segmented (fully mature) neutrophils)+the bands (almost mature neutrophils).

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in a reduction in the occurrence of severe (Grade 4) neutropenia (DSN) in patients compared to those receiving the sacituzumab govitecan chemotherapy protocol without trilaciclib. Severe neutropenia is defined as the absolute neutrophil count (ANC) laboratory value that meets the National Cancer Institute Common Terminology Criteria for Adverse Events (NCI CTCAE) v5.0 criteria for ≥Grade 4 toxicity (i.e., ANC <0.5×10⁹/L in SI Unit). Severe neutropenia is generally quantified as an absolute neutrophil count (ANC) of less than 500 per microliter (500/μL). Methods of calculating absolute neutrophil count (ANC) are well known in the art and include multiplying the WBC count times the percent of neutrophils in the differential WBC count. The percent of neutrophils consists of the segmented (fully mature) neutrophils+the bands (almost mature neutrophils).

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in a reduction in the duration of severe (Grade 4) neutropenia (DSN) in patients compared to those receiving the sacituzumab govitecan-chemotherapy protocol without trilaciclib. The duration of DSN is generally defined as the number of days from the date of the first ANC value of <0.5×10⁹/L to the date of the first ANC value ≥0.5×10⁹/L where no additional ANC values <0.5×10⁹/L are observed for the remainder of that cycle. Severe neutropenia is generally quantified as an absolute neutrophil count (ANC) of less than 500 per microliter (500/μL). Methods of calculating absolute neutrophil count (ANC) are well known in the art and include multiplying the WBC count times the percent of neutrophils in the differential WBC count. The percent of neutrophils consists of the segmented (fully mature) neutrophils+the bands (almost mature neutrophils).

In some embodiments, the inclusion of trilaciclib in a sacituzumab govitecan-chemotherapy protocol described herein for the treatment of advanced or metastatic TNBC, or alternatively advanced or metastatic urothelial cancer, or still alternatively a Trop-2 overexpressing cancer including but not limited to NSCLC results in a reduction of chemotherapy-induced fatigue (CIF) in patients compared to those receiving sacituzumab govitecan chemotherapy protocol without trilaciclib. In some embodiments, the reduction of CIF is a reduction in the time to first confirmed deterioration of fatigue (TTCD-fatigue), as measured by the Functional Assessment of Cancer Therapy-Fatigue (FACIT-F). FACIT-F is a 13-item subscale that measures fatigue severity and the impact of fatigue on functioning and is described in Yellen et al., Measuring fatigue and other anemia-related symptoms with the Functional Assessment of Cancer Therapy (FACT) measurement system. J Pain Symptom Manage. 1997; 13: 63-74.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in a reduction in severe neutropenia events, a reduction in granulocyte-colony stimulating factor (G-CSF) treatment, or a reduction in febrile neutropenia (FN) adverse events (AEs) in patients compared to those receiving sacituzumab govitecan-hziy chemotherapy protocol without trilaciclib. G-CSF treatment will be utilized according to the treatment guidelines outlined in Aapro et al. 2010 update of EORTC guidelines for the use of granulocyte-colony stimulating factor to reduce the incidence of chemotherapy-induced febrile neutropenia in adult patients with lymphoproliferative disorders and solid tumours. Eur J Cancer. 2011; 47:8-32.

In some embodiments, the inclusion of trilaciclib in a sacituzumab govitecan chemotherapy protocol described herein for the treatment of advanced or metastatic TNBC or alternatively advanced or metastatic urothelial cancer, or still alternatively a Trop-2 overexpressing cancer including but not limited to NSCLC results in a reduction in Grade 3 or 4 decreased hemoglobin laboratory values, red blood cell (RBC) transfusions, or erythropoiesis-stimulating agent (ESA) administration.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in a reduction in Grade 3 or 4 decreased platelet count laboratory values and/or the number of platelet transfusions. As described in (Kaufman, 2015; Schiffer, 2017), platelets are generally transfused at a threshold of ≤10,000/μL. Platelets are also generally transfused in any patient who is bleeding with a platelet count <50,000/μL (100,000/μL for central nervous system or ocular bleeding).

In some embodiments, the inclusion of trilaciclib in a sacituzumab govitecan chemotherapy protocol described herein for the treatment of advanced or metastatic TNBC or alternatively advanced or metastatic urothelial cancer, or still alternatively a Trop-2 overexpressing cancer including but not limited to NSCLC results in a reduction in Grade 3 or 4 hematologic laboratory values. In some embodiments, the use of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer results in a reduction in all-cause dose reductions or cycle delays and relative dose intensity of a sacituzumab govitecan chemotherapy protocol described herein.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in a reduction in i) hospitalizations, including but not limited to those due to all causes, febrile neutropenia/neutropenia, anemia/RBC transfusion, thrombocytopenia/bleeding and infections or ii) antibiotic use, including but not limited to intravenous (IV), oral, and oral and IV administered antibiotics.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC results in an improvement to one or more of: Functional Assessment of Cancer Therapy-General (FACT-G) domain scores (physical, social/family, emotional, and functional well-being); Functional Assessment of Cancer Therapy-Anemia (FACT-An); 5-level EQ-5D (EQ-5D-5L); Patient Global Impression of Change (PGIC) fatigue item; or Patient Global Impression of Severity (PGIS) fatigue item.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC, results in a reduction in the number of severe diarrhea episodes (Grade 3 or greater) experienced by a patient compared to those receiving the sacituzumab govitecan chemotherapy protocol described herein without trilaciclib.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC, results in a reduction in the development of, severity of, or episodes of mucositis experienced by a patient compared to those receiving sacituzumab govitecan chemotherapy protocol described herein without trilaciclib.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC, results in a reduction in the development of, severity of, or episodes of stomatitis experienced by a patient compared to those receiving sacituzumab govitecan chemotherapy protocol described herein without trilaciclib.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC, results in a reduction in the development of, severity of, alopecia experienced by a patient compared to those receiving sacituzumab govitecan chemotherapy protocol described herein without trilaciclib.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC, results in a reduction in the development of, severity of, gastrointestinal adverse events experienced by a patient compared to those receiving sacituzumab govitecan chemotherapy protocol described herein without trilaciclib.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC, results in a reduction in the number of severe anemia episodes (Grade 3 or greater) experienced by a patient compared to those receiving the sacituzumab govitecan chemotherapy protocol described herein without trilaciclib.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC, results in a reduction in the number of severe neutropenia episodes (Grade 3 or greater) experienced by a patient compared to those receiving the sacituzumab govitecan chemotherapy protocol described herein without trilaciclib.

In some embodiments, the inclusion of trilaciclib in a sacituzumab govitecan chemotherapy protocol described herein for the treatment of advanced or metastatic TNBC, or alternatively advanced or metastatic urothelial cancer, or still alternatively a Trop-2 overexpressing cancer including but not limited to NSCLC, results in a reduction in the number of febrile neutropenia episodes (Grade 3 or greater) experienced by a patient compared to those receiving the sacituzumab govitecan chemotherapy protocol described herein without trilaciclib.

### Pharmaceutical Compositions

The selected compounds of the protocols described herein or their pharmaceutically acceptable salts can be administered as the neat chemical, but is more typically administered as a pharmaceutical composition, that includes an effective amount for a patient, typically a human, in need of such treatment in a pharmaceutically acceptable carrier. The pharmaceutical composition may contain a compound or salt thereof as the only active agent, or, in an alternative embodiment, the compound or its salt and at least one additional active agent for the disease to be treated.

The pharmaceutical compositions may be administered in a therapeutically effective amount by any desired mode of administration, but is typically administered as an intravenous injection or infusion. In alternative embodiments, the compounds or pharmaceutically acceptable salts are delivered in an effective amount with a pharmaceutically acceptable carrier for oral delivery. As more general non-limiting examples, the pharmaceutical composition one suitable for oral (including buccal and sub-lingual), rectal, nasal, topical, transdermal, pulmonary, vaginal or parenteral (including intramuscular, intra-arterial, intrathecal, subcutaneous and intravenous), injections, inhalation or spray, intra-aortal, intracranial, subdermal, intraperitoneal, subcutaneous, or by other means of administration containing conventional pharmaceutically acceptable carriers.

Suitable dosage ranges depend upon numerous factors such as the severity of the disease to be treated, the age and relative health of the patient, the potency of the compound used, the route and form of administration, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

In certain embodiments the pharmaceutical composition is in a dosage form that contains from about 0.01 mg to about 1000 mg, from about 0.1 mg to about 750 mg, from about 1 mg to about 500 mg, or from about 5, 10, 15, or 20 mg to about 250 mg of the active compound or its pharmaceutically acceptable salt. Examples are dosage forms are those delivering at least 0.01, 0.05, 0.1, 1, 5, 10, 25, 50, 100, 200, 250, 300, 400, 500, 600, 700, or 750 mg of active compound, or its salt. When the weight is used herein, it can refer to either the compound alone or the compound in combination with its pharmaceutically acceptable salt.

An effective amount of the disclosed compound or its salt may be administered based on the weight, size or age of the patient. For example, a therapeutic amount may for example be in the range of about 0.01 mg/kg to about 250 mg/kg body weight, or about 0.1 mg/kg to about 10 mg/kg, in at least one dose. The patient can be administered as many doses as are required to reduce and/or alleviate and/or cure the disorder in question. When desired, formulations can be prepared with enteric coatings adapted for sustained or controlled release administration of the active ingredient.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packed tablets, capsules, and powders in vials or ampoules. Also, the unit dosage form can be a capsule, tablet, cachet, or lozenge itself, or it can be the appropriate number of any of these in packaged form.

In certain embodiments the compound is administered as a pharmaceutically acceptable salt. Non-limiting examples of pharmaceutically acceptable salts include: acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Depending on the intended mode of administration, the pharmaceutical compositions can be in the form of solid, semi-solid or liquid dosage forms, such as, for example, tablets, suppositories, pills, capsules, powders, liquids, syrup, suspensions, creams, ointments, lotions, paste, gel, spray, aerosol, foam, or oil, injection or infusion solution, a transdermal patch, a subcutaneous patch, an inhalation formulation, in a medical device, suppository, buccal, or sublingual formulation, parenteral formulation, or an ophthalmic solution, or the like, preferably in unit dosage form suitable for single administration of a precise dosage.

Some dosage forms, such as tablets and capsules, are subdivided into suitably sized unit doses containing appropriate quantities of the active components, e.g., an effective amount to achieve the desired purpose. The compositions will include an effective amount of the selected drug in combination with a pharmaceutically acceptable carrier and, in addition, can include other pharmaceutical agents, adjuvants, diluents, buffers, and the like.

Carriers include excipients and diluents and should be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to adjuvants, binders, buffering agents, coloring agents, diluents, disintegrants, excipients, emulsifiers, flavorants, gels, glidents, lubricants, preservatives, stabilizers, surfactants, solubilizer, tableting agents, wetting agents or solidifying material.

Some carriers may be listed in more than one class, for example vegetable oil may be used as a lubricant in some formulations and a diluent in others.

Exemplary pharmaceutically acceptable carriers include sugars, starches, celluloses, powdered tragacanth, malt, gelatin; talc, petroleum jelly, lanoline, polyethylene glycols, alcohols, transdermal enhancers and vegetable oils. Optional active agents may be included in a pharmaceutical composition, which do not substantially interfere with the activity of the compound of the present invention.

Some excipients include, but are not limited, to liquids such as water, saline, glycerol, polyethylene glycol, hyaluronic acid, ethanol, and the like. The compound can be provided, for example, in the form of a solid, a liquid, spray dried material, a microparticle, nanoparticle, controlled release system, etc., as desired according to the goal of the therapy. Suitable excipients for non-liquid formulations are also known to those of skill in the art. A thorough discussion of pharmaceutically acceptable excipients and salts is available in Remington' s Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline, and the like.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington' s Pharmaceutical Sciences, referenced above.

In yet another embodiment provided is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

In certain embodiments the excipient is selected from butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The pharmaceutical compositions containing the active agents can be formulated for oral administration. For oral administration, the composition may take the form of a tablet, capsule, a softgel capsule or can be an aqueous or nonaqueous solution, suspension or syrup. Tablets and capsules are typical oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Typically, sterile injectable suspensions are formulated according to techniques known in the art using suitable carriers, dispersing or wetting agents and suspending agents. The sterile injectable formulation can also be a sterile injectable solution or a suspension in an acceptably nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

The claimed invention is further described by way of the following non-limiting examples. Further aspects and embodiments of the present invention will be apparent to those of ordinary skill in the art, in view of the above disclosure and following experimental exemplification, included by way of illustration and not limitation, and with reference to the attached figures.

### EXAMPLES

### Example 1

### A Phase 2, Single-Arm, Open-Label Study of Trilaciclib Administered Prior to Sacituzumab Govitecan-Hziy in Patients with Unresectable Locally Advanced or Metastatic Triple-Negative Breast Cancer who Received at least Two Prior Treatments, at Least One in the Metastatic Setting

### Overview

An exploratory Phase 2, multicenter, open-label study evaluating the safety and efficacy of trilaciclib administered with sacituzumab govitecan-hziy in patients with unresectable locally advanced or metastatic TNBC who received at least 2 prior treatments, at least 1 in the metastatic setting, is provided herein.

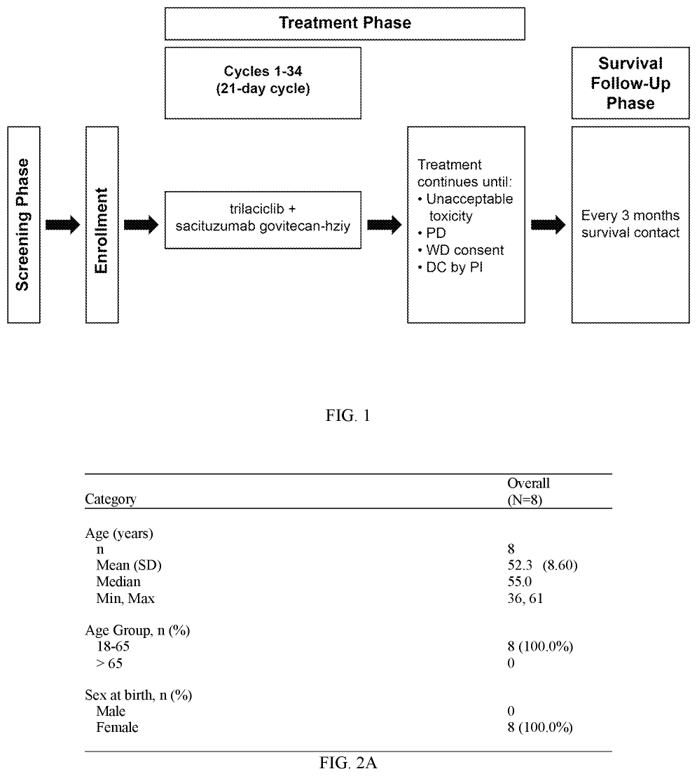

A general schematic describing the clinical trial is provided in FIG. 1. Approximately 45 patients will be enrolled in this study.

Trilaciclib plus sacituzumab govitecan-hziy will be administered intravenously (IV) in 21-day cycles as follows:
Trilaciclib 240 mg/m² administered as a 30-minute IV infusion completed within 4 hours prior to the start of sacituzumab govitecan-hziy on Day 1 and Day 8 of each 21-day treatment cycle.
Sacituzumab govitecan-hziy 10 mg/kg administered IV on Day 1 and Day 8 of each 21-day treatment cycle.

The first infusion of sacituzumab govitecan-hziy is administered over 3 hours and the patient is observed during the infusion and for at least 30 minutes following the initial dose for signs or symptoms of infusion-related reactions. Subsequent infusions of sacituzumab govitecan-hziy may be administered over 1 to 2 hours if prior infusions were tolerated and patients should be observed during the infusion and for at least 30 minutes after infusion. Prior to each infusion of sacituzumab govitecan-hziy, premedication for the prevention of infusion reactions and chemotherapy-induced nausea and vomiting (CINV) is recommended.
Premedicate with antipyretics, histamine receptor 1 (H1) and histamine receptor 2 (H2) blockers prior to infusion, and corticosteroids may be used for patients who had prior infusion reactions.
Premedicate with a two or three drug combination regimen (e.g., dexamethasone with either a 5-HT3 receptor antagonist or a neurokinin 1 (NK1) receptor antagonist, as well as other drugs as indicated).

The study will include 3 study phases: Screening Phase, Treatment Phase, and Survival Follow up Phase (See FIG. 1). The Treatment Phase begins on the day of the first dose of study treatment and completes at the Safety Follow-up Visit. Survival Follow-up assessments should occur approximately every 3 months after the End of Treatment Visit.

Patients enrolled in the study will be eligible to receive treatment until disease progression, unacceptable toxicity, withdrawal of consent, investigator decision, or the end of the trial, whichever comes first. Treatment cycles will occur consecutively without interruption, except when necessary to manage toxicities or for administrative reasons. A 3-week delay from the scheduled dose of sacituzumab govitecan-hziy is permitted. As examples, if at the scheduled Cycle X Day 1 visit (e.g., Cycle 2 Day 1) a dose delay is needed, a delay up to 3 weeks from Cycle X Day 1 for sacituzumab govitecan-hziy is permitted; if the scheduled Cycle X Day 8 visit (Cycle 1 Day 8) a dose delay is needed, a delay up to 3 weeks from Cycle X Day 1 for sacituzumab govitecan-hziy is permitted. A dosing delay >3 weeks from the scheduled dose of sacituzumab govitecan-hziy may be permitted on a case-by-case basis with the approval of the Investigator and Medical Monitor.

An End of Treatment Visit will occur approximately 14 days following a patient's last dose of study treatment. Safety Follow-up visits (which may be a phone call) will occur 30 days after the last dose of study treatment. Patients will be followed for survival approximately every 3 months after the End of Treatment Visit. Survival Follow-up Visits may be done via telephone, email, or clinic visit. Unless otherwise decided by the Sponsor, the study will continue until at least 70% of patients enrolled in the study have died.

### Diagnosis and Main Eligibility Criteria

Patients ≥18 years of age at the time of signing the informed consent with measurable locally advanced, unresectable, or metastatic TNBC (defined as <1% estrogen receptor [ER] and progesterone receptor by immunohistochemistry [IHC], human epidermal growth factor receptor 2 [HER2]-negative by IHC or in situ hybridization [ISH]) and with an Eastern Cooperative Oncology Group (ECOG) performance status of 0 or 1. Patients must have measurable disease as defined by RECIST v1.1 and considered eligible to receive sacituzumab govitecan-hziy treatment. Patients with known brain metastasis at the time of enrollment are not eligible. Patients must be refractory to or relapsed after at least 2 prior standard of care chemotherapy regimens for unresectable, locally advanced or metastatic breast cancer (these regimens will qualify regardless of TNBC status at the time they were administered. There is no upper limit in the number of prior therapies for locally advanced or metastatic disease. Adjuvant or neoadjuvant treatment for more limited disease will qualify as one of the required prior regimens if the development of unresectable, locally advanced or metastatic disease occurred within a 12-month period of time after completion of chemotherapy. For patients with a documented germ-line BRCAl/BRCA2 mutation who received an approved PARP inhibitor, the PARP inhibitor can be used to meet the criteria for 1 of the 2 prior standard of care chemotherapies. All patients must have prior taxane treatment in either the neoadjuvant, adjuvant, or advanced/metastatic setting. Prior radiation therapy is permitted for recurrent disease as long as the patient has at least 1 measurable lesion that his not been irradiated. Patients must also have adequate organ function as demonstrated by laboratory values.

### Dose, Dosing Regimen, and Route Trilaciclib

Trilaciclib for Injection, 300 mg/vial (also referred to as “Trilaciclib Sterile Powder for concentrate for solution for IV infusion, 300 mg/vial”) is supplied as a sterile, preservative free, yellow, lyophilized cake in a single-dose vial (300 mg/20 mL).

Trilaciclib must be reconstituted and further diluted prior to IV infusion. Upon reconstitution, the solution must then be diluted to the calculated dose (240 mg/m²) based on the body surface area (BSA) of the patient. Actual body weight (not ideal body weight) should be utilized for dose calculations.

Administer diluted trilaciclib solution as a 30-minute IV infusion no more than 4 hours prior to sacituzumab govitecan-hziy. Do not administer trilaciclib as a bolus. Trilaciclib is always administered first. Results from hematology labs should be reviewed prior to administration of trilaciclib. If administration of sacituzumab govitecan-hziy therapy is skipped or discontinued, trilaciclib will also be skipped or discontinued.

### Sacituzumab Govitecan-Hziy (Trodelvy®)

Descriptions of the formulations of commercially-available sacituzumab govitecan-hziy can be found in the respective current prescribing information. Protocol specified doses of sacituzumab govitecan-hziy will be administered via IV in accordance with institutional guidelines according to the study site's standard practice.

Actual body weight (not ideal body weight) should be utilized for dose calculations. At a minimum, if there is a change in body weight of >10% relative to the weight at the time of the last dose calculation, doses should be recalculated. Recalculation of the dose more frequently per local institutional guidelines is permitted. Dose recalculation to adjust for changes in body weight will not be considered a dose reduction.

Prior to each infusion of sacituzumab govitecan-hziy, premedication for the prevention of infusion reactions and CINV is recommended.
Premedicate with antipyretics, H1 and H2 blockers prior to infusion, and corticosteroids may be used for patients who had prior infusion reactions.

Premedicate with a two or three drug combination regimen (e.g., dexamethasone with either a 5-HT3 receptor antagonist or a NK1 receptor antagonist, as well as other drugs as indicated).

Administer the first infusion over 3 hours and observe patients during the infusion and for at least 30 minutes following the initial dose for signs or symptoms of infusion-related reactions.

Subsequent infusions may be administered over 1 to 2 hours if prior infusions were tolerated and patients should be observed during the infusion and for at least 30 minutes after infusion. Protect infusion bag from light. Do not administer as an IV push or bolus.

Trilaciclib is always administered first, followed by sacituzumab govitecan-hziy. Administer diluted trilaciclib solution as a 30-minute IV infusion to be completed within 4 hours prior to the start of sacituzumab govitecan-hziy. If administration of trilaciclib is delayed or discontinued, sacituzumab govitecan-hziy will also be delayed or discontinued. Likewise, if sacituzumab govitecan-hziy is delayed or discontinued, trilaciclib will also be delayed or discontinued.

### Criteria for Starting Cycle 1 and Each Subsequent Cycle

To start the treatment, patients must meet all of the following criteria to receive study treatment on Cycle 1, Day 1:
ANC ≥1.5×10⁹/L;
Platelet count ≥100×10⁹/L;
Total bilirubin ≤1.5×ULN; and,
AST/ALT ≤3×ULN or ≤5×ULN in presence of liver metastasis.
Other nonhematologic drug-related toxicities must be ≤Grade 1 (except alopecia or peripheral neuropathy that may be Grade 2 or less).

Patients must also have ANC ≥1.5×10⁹/L to receive any subsequent Cycle Day 1 dose of study treatment, and ANC ≥1.0×10⁹/L to receive any Cycle Day 8 dose of study treatment.

For dose delays due to toxicity, the patient should be followed (at least) weekly, including CBCs if the AE is hematologic, to monitor the toxicity until treatment criteria are met or until the patient discontinues treatment (e.g., if more than 3 weeks elapse from the last treatment dose).

A 3-week delay from the scheduled dose of sacituzumab govitecan-hziy is permitted for toxicity and/or administrative reasons. As examples, if at the scheduled Cycle X Day 1 visit (e.g., Cycle 2 Day 1) a dose delay is needed for toxicity reasons, a delay up to 3 weeks from Cycle X Day 1 for sacituzumab govitecan-hziy is permitted; if at the scheduled Cycle X Day 8 visit (e.g., Cycle 1 Day 8) a dose delay is needed for toxicity reasons, a delay up to 3 weeks from Cycle X Day 1 for sacituzumab govitecan-hziy is permitted. Dosing delays >3 weeks from the scheduled dose of sacituzumab govitecan-hziy may be permitted on a case by-case basis with the documented approval of the Investigator and Medical Monitor.

Study drug administration will continue until progressive disease per RECIST v1.1 or clinical progression as determined by the investigator, unacceptable toxicity, withdrawal of consent, investigator decision, the patient has received a maximum of 34 cycles of treatment, or the end of the study, whichever occurs first.

### Criteria for Evaluation

Efficacy: Anti-tumor efficacy assessments include progression free survival (PFS), objective response rate (ORR), clinical benefit rate (CBR), duration of response (DOR) and overall survival (OS). Tumor response criteria will be based on RECIST v1.1.

Myelosuppression endpoints will be based on reported hematology assessments, myelosuppression-related adverse event (AE) details, dose reductions/delays, and supportive care interventions (including transfusions).

Safety: Safety will be evaluated by monitoring AEs, clinical laboratory test results (hematology, clinical chemistry), vital sign measurements (blood pressure, heart rate, and oral body temperature), 12 lead safety electrocardiogram (ECG) results, dose modifications, and physical examination findings.

### Results:

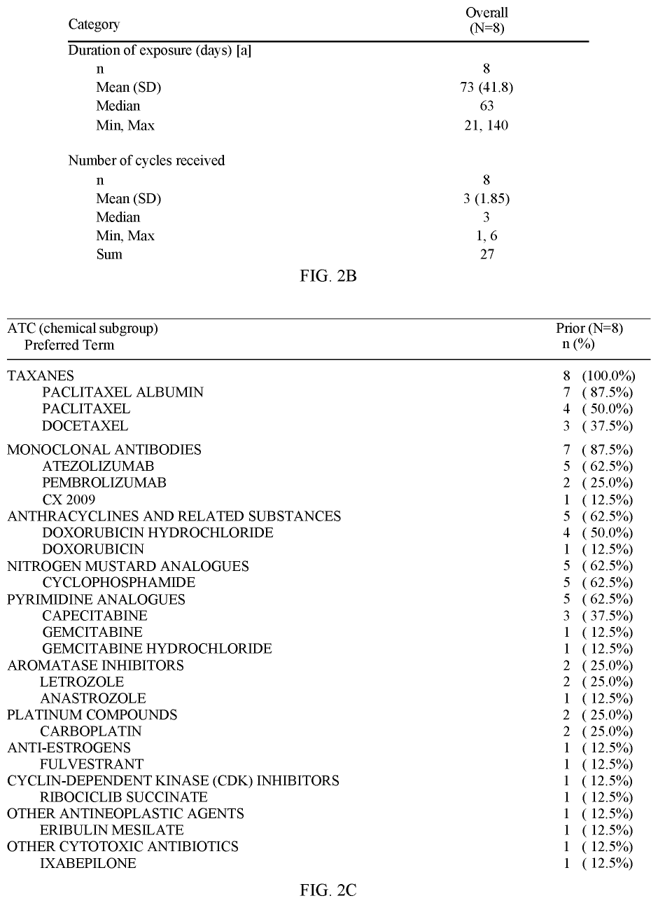

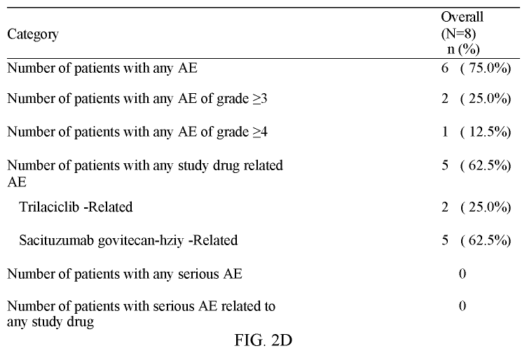

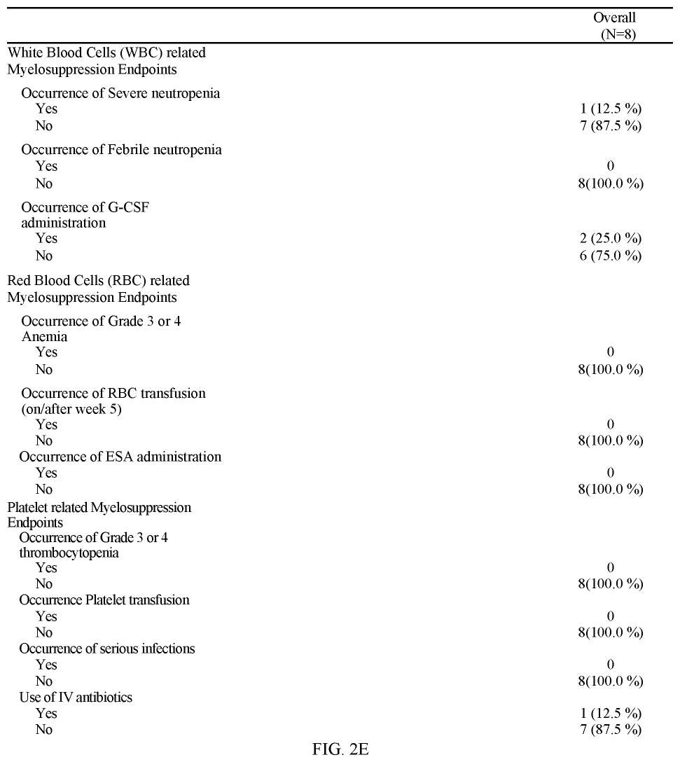

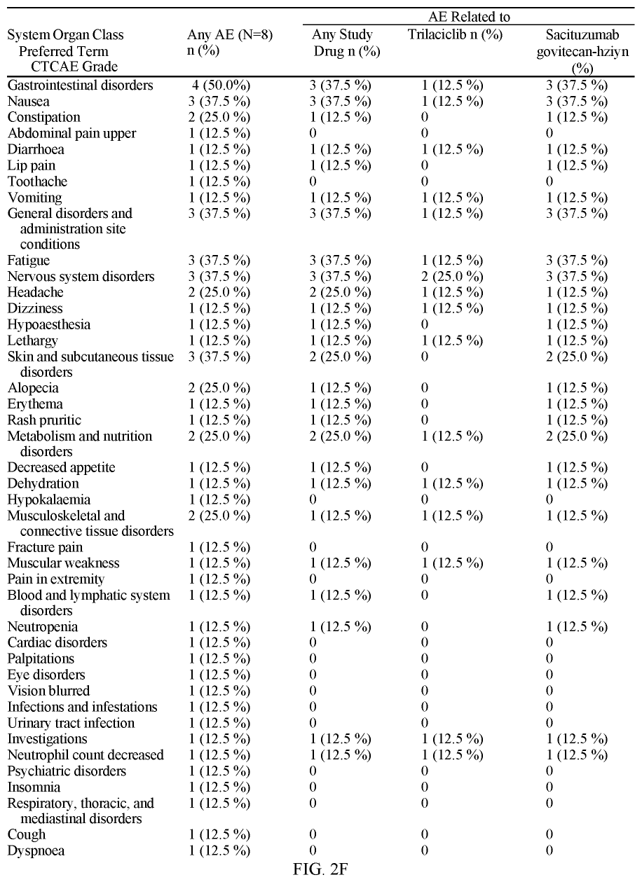

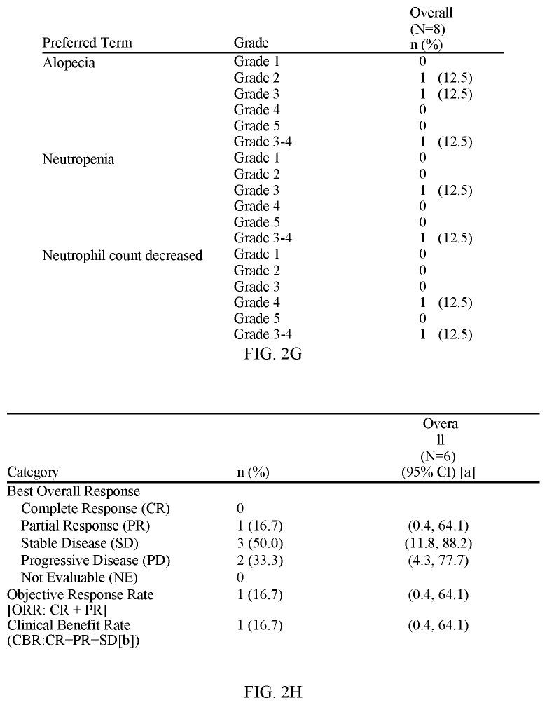

As of Jun. 3, 2022, 8 female patients (median age 55.0 years) (FIG. 2A) had been enrolled and completed a median 3 (range 1-6) 21-day cycles (FIG. 2B). All patients had received a prior taxane and 7 had received a prior immune checkpoint inhibitor (5 atezolizumab, 2 pembrolizumab; 4 in the adjuvant setting, 3 in the metastatic setting) (FIG. 2C). Overall, 5 patients had ≥1 treatment-related adverse event (TRAE), including 2 patients with trilaciclib-related AEs and 5 patients with sacituzumab govitecan-related AEs (FIG. 2D). No patients were withdrawn due to an AE and no serious AEs have been reported to date. Severe (Grade 3/4) neutropenia occurred in 1 patient (FIG. 2E). No patients had Grade 3/4 anemia or thrombocytopenia, and none had febrile neutropenia or a serious infection. Granulocyte colony-stimulating factors were administered to 2 patients and 1 patient required intravenous antibiotics. No patients required red blood cell/platelet transfusions or erythropoiesis-stimulating agents. The most common TRAEs were nausea (n=3), fatigue (n=3), and headache (n=2) (FIG. 2F). There was 1 TRAE of diarrhea (trilaciclib- and sacituzumab govitecan-related). Two patients had Grade ≥3 TRAEs, comprising Grade 3 alopecia and Grade 3 neutropenia (both sacituzumab govitecan-related), and Grade 4 neutrophil count decreased (trilaciclib- and sacituzumab govitecan-related) (FIG. 2G). At present, one patient has demonstrated a partial response and three patients have experienced stable disease (FIG. 2H).

Preliminary data are encouraging and indicate that trilaciclib administered prior to sacituzumab govitecan is well tolerated and may provide clinical benefits in patients with mTNBC who have received ≥2 prior systemic therapies.

What is claimed is:

1. A method of treating a patient having advanced or metastatic triple negative breast cancer (TNBC) comprising:
i) administering to the patient an effective amount of a cyclin-dependent kinase 4/6 (CDK4/6) inhibitor of structure: 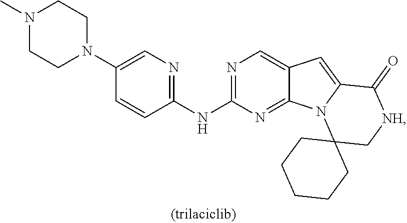 [SMILES: CN1CCN(c2ccc(Nc3ncc4cc5n(c4n3)C3(CCCCC3)CNC5=O)nc2)CC1]
or a pharmaceutically acceptable salt, composition, isotope, or prodrug thereof; and
ii) administering to the patient an effective amount of sacituzumab govitecan;
wherein trilaciclib is administered about 4 hours or less prior to the initiation of administration of the sacituzumab govitecan.

2. The method of claim 1, wherein trilaciclib is administered about one hour or less prior to administration of sacituzumab govitecan.

3. The method of claim 1, wherein trilaciclib is administered to the patient intravenously at a dosage of between about 190 and 280 mg/m².

4. The method of claim 3, wherein trilaciclib is administered at about 240 mg/m².

5. The method of claim 1, wherein sacituzumab govitecan is administered at a dose of between about 5 mg/kg and 15 mg/kg.

6. The method of claim 5, wherein sacituzumab govitecan is administered at a dose of about 10 mg/kg.

7. The method of claim 1, wherein the method comprises one or more 21-day therapeutic cycles, wherein trilaciclib and sacituzumab govitecan are administered on days 1 and 8 of each 21-day cycle.

8. The method of claim 7, wherein the 21-day cycle is administered 2 to 35 times.

9. The method of claim 1, wherein the patient's TNBC is PD-L1 positive.

10. The method of claim 1, wherein the patient, prior to receiving trilaciclib and sacituzumab govitecan, previously received at least two lines of chemotherapeutic treatment.

11. The method of claim 10, wherein at least one of the previously received chemotherapeutic lines of treatment was in the metastatic setting.

12. The method of claim 10, wherein at least one of the previously received chemotherapeutic lines of treatment was with a taxane.

13. The method of claim 10, wherein at least one of the previously received chemotherapeutic lines of treatment was with a platinum compound.

14. The method of claim 10, wherein at least one of the previously received chemotherapeutic lines of treatment was with a PD-1 inhibitor or PD-L1 inhibitor.

15. The method of claim 1, wherein the TNBC is unresectable, locally advanced TNBC.

16. The method of claim 1, wherein the TNBC is metastatic TNBC.

17. The method of claim 1, wherein the administration of trilaciclib/sacituzumab govitecan regimen results in a reduction in the number of severe neutropenia episodes (Grade 3 or greater) experienced by a patient compared to those receiving sacituzumab govitecan chemotherapy without trilaciclib.

18. The method of claim 1, wherein the administration of trilaciclib/sacituzumab govitecan regimen results in a reduction in the number of severe diarrhea episodes (Grade 3 or greater) experienced by a patient compared to those receiving sacituzumab govitecan chemotherapy without trilaciclib.

19. The method of claim 1, wherein the treatment results in improved progression free survival (PFS) in patients compared to those receiving sacituzumab govitecan chemotherapy without trilaciclib.

20. The method of claim 1, wherein the treatment results in improved overall survival (OS) in patients compared to those receiving sacituzumab govitecan chemotherapy without trilaciclib.

21. The method of claim 1, wherein the sacituzumab govitecan administered is sacituzumab govitecan-hziy.
